SEQUENCE LISTING (63 sequences)

SEQ ID NO: 1 - DNA, 74 bp, Artificial Sequence
  Description of Artificial Sequence Synthetic oligonucleotide
cagtccggatgctctagagtgacnnnnnnnnnnnnnnnnnnnnnnnnnnnnnncgaatctcgtgaagccgagcg

SEQ ID NO: 2 - DNA, 23 bp, Artificial Sequence
  Description of Artificial Sequence Synthetic oligonucleotide primer
cagtccggatgctctagagtgac

SEQ ID NO: 3 - DNA, 21 bp, Artificial Sequence
  Description of Artificial Sequence Synthetic oligonucleotide primer
cgctcggcttcacgagattcg

SEQ ID NO: 4 - DNA, 30 bp, Artificial Sequence
  Description of Artificial Sequence Synthetic oligonucleotide
tgtcgttgtgtcctgtacccgccttgacca

SEQ ID NO: 5 - DNA, 30 bp, Artificial Sequence
  Description of Artificial Sequence Synthetic oligonucleotide
tgtctcgatgctagactctatacccgccaa

SEQ ID NO: 6 - DNA, 30 bp, Artificial Sequence
  Description of Artificial Sequence Synthetic oligonucleotide
tgtggactggtctatccatgcacctgtacc

SEQ ID NO: 7 - DNA, 30 bp, Artificial Sequence
  Description of Artificial Sequence Synthetic oligonucleotide
tgtgtgtatggtccttgcatcgattccctg

SEQ ID NO: 8 - DNA, 29 bp, Artificial Sequence
  Description of Artificial Sequence Synthetic oligonucleotide
tgtgtctatcgctgcaccgtgtccataca

SEQ ID NO: 9 - DNA, 30 bp, Artificial Sequence
  Description of Artificial Sequence Synthetic oligonucleotide
gtgtgcgttgtctgtccgtttcctgtccac

SEQ ID NO: 10 - DNA, 30 bp, Artificial Sequence
  Description of Artificial Sequence Synthetic oligonucleotide
tgtggcgttgatatcgactgtccttgccac

SEQ ID NO: 11 - DNA, 30 bp, Artificial Sequence
  Description of Artificial Sequence Synthetic oligonucleotide
ttggttgatgtgcatcgctgttcctgtcca

SEQ ID NO: 12 - DNA, 30 bp, Artificial Sequence
  Description of Artificial Sequence Synthetic oligonucleotide
tgggtcgctgatcgcatcgatactctccac

SEQ ID NO: 13 - DNA, 30 bp, Artificial Sequence
  Description of Artificial Sequence Synthetic oligonucleotide
atcgtcgactgtcctgtcactgtccatcca

SEQ ID NO: 14 - DNA, 30 bp, Artificial Sequence
  Description of Artificial Sequence Synthetic oligonucleotide
tggaggtgcctggatatatcgactcgaccc

SEQ ID NO: 15 - DNA, 30 bp, Artificial Sequence
  Description of Artificial Sequence Synthetic oligonucleotide
gtgtccttgtctagcctcgatacacgacac

SEQ ID NO: 16 - DNA, 30 bp, Artificial Sequence
  Description of Artificial Sequence Synthetic oligonucleotide
tgtggcgttgactgtacacgtcgatacacc

SEQ ID NO: 17 - DNA, 30 bp, Artificial Sequence
  Description of Artificial Sequence Synthetic oligonucleotide
tggctggtcactgtactctctgctctccac

SEQ ID NO: 18 - DNA, 30 bp, Artificial Sequence
  Description of Artificial Sequence Synthetic oligonucleotide
tgtagtgtcctggctatccacgtctccatt

SEQ ID NO: 19 - DNA, 30 bp, Artificial Sequence
  Description of Artificial Sequence Synthetic oligonucleotide
tggatcgcttatcgcctcgatcattgccca

SEQ ID NO: 20 - DNA, 30 bp, Artificial Sequence
  Description of Artificial Sequence Synthetic oligonucleotide
ttgttgtactggcatcgcctcgactcgctg

SEQ ID NO: 21 - DNA, 41 bp, Artificial Sequence
  Description of Artificial Sequence consensus oligonucleotide sequence
nntgtnganntgnnngtcnnctgtancngnnnctngaccnn

SEQ ID NO: 22 - protein, 6 aa, Artificial Sequence
  Description of Artificial Sequence Synthetic peptide
SGSGSG

SEQ ID NO: 23 - DNA, 14 bp, Artificial Sequence
  Description of Artificial Sequence Synthetic polyC oligonucleotide
cccccccccccccc

SEQ ID NO: 24 - DNA, 30 bp, Artificial Sequence
  Description of Artificial Sequence Synthetic oligonucleotide
aaaggaaacgtgaatcgacacgtcaccaca

SEQ ID NO: 25 - DNA, 30 bp, Artificial Sequence
  Description of Artificial Sequence Synthetic oligonucleotide
aaggggacaggcaatggacacgtcaccaca

SEQ ID NO: 26 - DNA, 30 bp, Artificial Sequence
  Description of Artificial Sequence Synthetic oligonucleotide
tggtcaaggcgggtacaggacacaacgaca

SEQ ID NO: 27 - DNA, 30 bp, Artificial Sequence
  Description of Artificial Sequence Synthetic oligonucleotide
gtgaaagtgcaatggatccaggaccccaca

SEQ ID NO: 28 - DNA, 30 bp, Artificial Sequence
  Description of Artificial Sequence Synthetic oligonucleotide
gtggcaagggaaccgatacgcgactctcca

SEQ ID NO: 29 - DNA, 20 bp, Artificial Sequence
  Description of Artificial Sequence Synthetic oligonucleotide
aggtcaaggaacagtaccag

SEQ ID NO: 30 - DNA, 29 bp, Artificial Sequence
  Description of Artificial Sequence Synthetic oligonucleotide
tggagaggcggggaagtctatgcaccact

SEQ ID NO: 31 - DNA, 30 bp, Artificial Sequence
  Description of Artificial Sequence Synthetic oligonucleotide
gtggatagcaggggagtacaggcaacacca

SEQ ID NO: 32 - DNA, 30 bp, Artificial Sequence
  Description of Artificial Sequence Synthetic oligonucleotide
aagggaacagcaaggacagacatgtcccac

SEQ ID NO: 33 - DNA, 30 bp, Artificial Sequence
  Description of Artificial Sequence Synthetic oligonucleotide
gtgacgggaatcgatgcacagagccataca

SEQ ID NO: 34 - DNA, 30 bp, Artificial Sequence
  Description of Artificial Sequence Synthetic oligonucleotide
acggggctatactgtatccacggcaccacc

SEQ ID NO: 35 - DNA, 21 bp, Artificial Sequence
  Description of Artificial Sequence Synthetic oligonucleotide
ctgtacaggctagactcgacc

SEQ ID NO: 36 - DNA, 30 bp, Artificial Sequence
  Description of Artificial Sequence Synthetic oligonucleotide
aggagaagtggcaaggcaactgacagtcca

SEQ ID NO: 37 - DNA, 30 bp, Artificial Sequence
  Description of Artificial Sequence Synthetic oligonucleotide
gggtcaaggatcgatacaaggctccatcca

SEQ ID NO: 38 - DNA, 29 bp, Artificial Sequence
  Description of Artificial Sequence Synthetic oligonucleotide
aggcagagcagcgattcaatgccacaacg

SEQ ID NO: 39 - DNA, 30 bp, Artificial Sequence
  Description of Artificial Sequence Synthetic oligonucleotide
agggtagcagtgcagtgcagtgaagcgaca

SEQ ID NO: 40 - DNA, 30 bp, Artificial Sequence
  Description of Artificial Sequence Synthetic oligonucleotide
ggtaggcaaggatcgacgtgaacttcaccg

SEQ ID NO: 41 - DNA, 30 bp, Artificial Sequence
  Description of Artificial Sequence Synthetic oligonucleotide
ggtgagggtacatgtacacatcgactcaca

SEQ ID NO: 42 - DNA, 30 bp, Artificial Sequence
  Description of Artificial Sequence Synthetic oligonucleotide
ataggaggtgcatggacacggcgatgacca

SEQ ID NO: 43 - DNA, 29 bp, Artificial Sequence
  Description of Artificial Sequence Synthetic oligonucleotide
caggtaggctaggagattacatcgacacc

SEQ ID NO: 44 - DNA, 30 bp, Artificial Sequence
  Description of Artificial Sequence Synthetic oligonucleotide
aagcctaggggagtaatacaacgggcaacg

SEQ ID NO: 45 - DNA, 30 bp, Artificial Sequence
  Description of Artificial Sequence Synthetic oligonucleotide
aggtaaaaggcctgggtgtcaatgtccaca

SEQ ID NO: 46 - DNA, 30 bp, Artificial Sequence
  Description of Artificial Sequence Synthetic oligonucleotide
atggggtgagtagctgtacagctagacaca

SEQ ID NO: 47 - DNA, 30 bp, Artificial Sequence
  Description of Artificial Sequence Synthetic oligonucleotide
ggtgtagacagggagcagtgcagctaccag

SEQ ID NO: 48 - DNA, 25 bp, Artificial Sequence
  Description of Artificial Sequence Synthetic oligonucleotide
gtggagaggacagcgacgctatcca

SEQ ID NO: 49 - DNA, 30 bp, Artificial Sequence
  Description of Artificial Sequence Synthetic oligonucleotide
gtgaatgggcgatgtagccttgccacgaca

SEQ ID NO: 50 - DNA, 30 bp, Artificial Sequence
  Description of Artificial Sequence Synthetic oligonucleotide
gtgatacgagacaagcgtagcctcgaccca

SEQ ID NO: 51 - DNA, 30 bp, Artificial Sequence
  Description of Artificial Sequence Synthetic oligonucleotide
cagggagatacatggccgagatagacacag

SEQ ID NO: 52 - DNA, 30 bp, Artificial Sequence
  Description of Artificial Sequence Synthetic oligonucleotide
gtgaagggactcgatacgcgatgcacatcc

SEQ ID NO: 53 - DNA, 30 bp, Artificial Sequence
  Description of Artificial Sequence Synthetic oligonucleotide
agtgaagttggcagtgactgcatcgatgca

SEQ ID NO: 54 - DNA, 30 bp, Artificial Sequence
  Description of Artificial Sequence Synthetic oligonucleotide
cagtggagtatcgagtagagcctcgccaca

SEQ ID NO: 55 - DNA, 30 bp, Artificial Sequence
  Description of Artificial Sequence Synthetic oligonucleotide
aggtgaggttcagtgtccaatgtatcccca

SEQ ID NO: 56 - DNA, 30 bp, Artificial Sequence
  Description of Artificial Sequence Synthetic oligonucleotide
aagttgatatcatcgatgcaggtaaccgcc

SEQ ID NO: 57 - DNA, 30 bp, Artificial Sequence
  Description of Artificial Sequence Synthetic oligonucleotide
aggttacagtggatatcgatacgccaagcc

SEQ ID NO: 58 - DNA, 30 bp, Artificial Sequence
  Description of Artificial Sequence Synthetic oligonucleotide
tgaagagatatagctgagcagagaggtcac

SEQ ID NO: 59 - DNA, 30 bp, Artificial Sequence
  Description of Artificial Sequence Synthetic oligonucleotide
gtgacatggancttggggtngagatgccca

SEQ ID NO: 60 - DNA, 30 bp, Artificial Sequence
  Description of Artificial Sequence Synthetic oligonucleotide
gtgggcagtgtcgcggtgatgtcatcgacc

SEQ ID NO: 61 - DNA, 74 bp, Artificial Sequence
  Description of Artificial Sequence Synthetic oligonucleotide
cagtccggatgctctagagtgacggtgagggtacatgtacacatcgactcacacgaatctcgtgaagccgagcg

SEQ ID NO: 62 - DNA, 74 bp, Artificial Sequence
  Description of Artificial Sequence Synthetic oligonucleotide
cagtccggatgctctagagtgacgtgaaagtgcaatggatccaggaccccacacgaatctcgtgaagccgagcg

SEQ ID NO: 63 - DNA, 74 bp, Artificial Sequence
  Description of Artificial Sequence Synthetic oligonucleotide
cagtccggatgctctagagtgacaaggggacaggcaatggacacgtcaccacacgaatctcgtgaagccgagcg

(19) United States
(12) Patent Application Publication
(10) Pub. No.: US 2005/0239134 A1
(43) Pub. Date: Oct. 27, 2005

(54) Combinatorial selection of phosphorothioate single-stranded DNA aptamers for TGF-beta-1 protein

(75) Inventors: David G. Gorenstein, Houston, TX (US); Bruce A. Luxon, Galveston, TX (US); Jonghoon Kang, Galveston, TX (US); Myung Soog Lee, Galveston, TX (US); John Alton Copland III, Ponte Vedra Beach, FL (US)

Correspondence Address: CHALKER FLORES, LLP, 12700 PARK CENTRAL, STE. 455, DALLAS, TX, 75251, US

(73) Assignee: Board of Regents, The University of Texas System, Austin, TX (US)

(21) Appl. No.: 10/828,934

(22) Filed: Apr. 21, 2004

(51) Int. Cl. C12Q 1/68; G01N 33/53; A61K 38/17; C07K 14/71

(52) U.S. Cl. 435/7.1; 514/12; 530/350

(57) ABSTRACT

The present invention includes the selection and isolation of thioaptamers that target the signaling protein TGF-β1, compositions of such thioaptamers and the use of such thioaptamers to either block or enhance signal transduction of the TGF-β1 protein and thus function as, e.g., immunomodulatory agents. Thioaptamers may also be targeted alone or in combination with other thioaptamers against the ligand, the receptors, the ligand trap protein(s) and/or the co-receptors to modulate TGF-β signaling pathway.

### TECHNICAL FIELD OF THE INVENTION

The present invention relates in general to the field of partially thio-modified aptamers or thioaptamers, and more particularly, to thioaptamers for drug discovery, evaluation and characterization of physiological pathways that target TGF-β and/or the TGF-β receptor and related proteins and the development of therapeutic agents based thereon.

### BACKGROUND OF THE INVENTION

This application is a continuation-in-part and claims priority based on U.S. patent application Ser. No. 10/272,509, filed Oct. 16, 2002, which is a continuation-in-part of U.S. patent application Ser. No. 09/425,798, filed Oct. 25, 1999, now U.S. Pat. No. 6,423,493, which is a continuation-in-part of U.S. patent application Ser. No. 09/425,804, filed Oct. 25, 1999, which is a continuation-in-part of U.S. Provisional Application Ser. No. 60/105,600, filed Oct. 26, 1998. This work was supported by the following United States grants: NIH A127744—Combinatorial and rational design of aptamers targeting HIV, NHLBI N01-HV-28184—Proteomic Technologies to Study Airway Inflammation; and NIAID U01 AI054827—Biodefense Proteomics Collaboratory; the government may own certain rights. Without limiting the scope of the invention, its background is described in connection with oligonucleotide agents and with methods for the isolation of modified oligonucleotides that bind specifically to target proteins.

Virtually all organisms have nuclease enzymes that degrade rapidly foreign DNA as an important in vivo defense mechanism. The use, therefore, of normal oligonucleotides or aptamers as diagnostic or therapeutic agents in the presence of most bodily fluids or tissue samples is generally precluded. It has been shown, however, that phosphoromonothioate or phosphorodithioate modifications of the DNA backbone in oligonucleotides can impart both nuclease resistance and enhance the affinity for target molecules, such as for example the transcriptional regulating protein NF-κB. Therefore, there is a need in the art for methods for generating aptamers that have enhanced binding affinity for a target molecule, as well as retained specificity. Also needed are ways to identify and quantify in detail the mechanisms by which aptamers interact with target molecules.

Synthetic phosphodiester-modified oligonucleotides such as phosphorothioate oligonucleotide (S-ODN) and phosphorodithioate oligonucleotide (S₂-ODN) analogues have increased nuclease resistance and may bind to proteins with enhanced affinity. Unfortunately, ODNs possessing high fractions of phosphorothioate or phosphorodithioate linkages may lose some of their specificity and are “stickier” towards proteins in general than normal phosphate esters, an effect often attributed to non-specific interactions. The recognition of nucleic acid sequences by proteins involves specific side-chain and backbone interactions with both the nucleic acid bases as well as the phosphate ester backbone, effects which may be disrupted by the non-specific interactions caused with S-ODN and S₂-ODN analogues.

Other advances in combinatorial chemistry allow construction and screening of large random sequence nucleic acid “aptamer” libraries (e.g., Ellington, A. D. and Szostak, J. W. (1990)); targeting proteins (e.g., Bock, L. C., et al., (1992)); and other molecules (Koizumi, M. and Breaker, R. R. (2000); Gold, L., et al. (1997); and Ye, X., et al. (1996)). Such antisense phosphorothioate oligonucleotides have been used to study the role of TGF-β in such tumors and such antisense agents have been proposed as immunostimulants of T-cell production for treatment of gliomas, acting via inhibition of TGF-β expression (Jachimczak, et al., 1991; Jachimczak, et al., 1993; Jachimczak, et al., 1995; Jachimczak, et al., 1996).

U.S. Pat. No. 6,455,689, issued to Biognostik Gesellschaft fur Biomolekulare Diagnostik, teaches specific compositions of such antisense phosphorothioates developed by the Jachimczak/Bogdahn group. Another patent application by the same group (U.S. Patent Application No. 20030040499) shows the use of such antisense phosphorothioates to stimulate T-cell production in the treatment of breast cancer, neurofibromas and malignant gliomas. For example, AntiSense Pharma began a Phase I/II clinical trial of these TGF-β antisense phosphorothioate oligonucleotides in Germany in late 2000. Initial data indicates satisfactory safety and clinical efficacy in to blocking TGF-β2 expression in patients with malignant glioma (Hau, et al., 2002).

Antisense gene therapy based on a plasmid vector expressing unmodified antisense oligonucleotide specific to TGF-β2 was used to block TGF-β immunosuppression and increase survival in a rat 9L gliosarcoma model (Fakhrai, et al., 1996). A series of patents teaching the use of TGF-β antisense gene therapy have issued to the Sidney Kimmel Cancer Center of San Diego (U.S. Pat. Nos. 5,772,995; 6,120,763; and 6,447,769). NovaRx Corp. is conducting currently a Phase II clinical trial of the TGF-β2 antisense gene therapy, in patients with lung cancer.

Whereas both antisense phosphorothioate oligonucleotides and gene therapy expressing antisense unmodified oligonucleotides modulate TGF-β activity by blocking gene expression of the TGF-β protein, alternative approaches in which TGF-β activity is modulated following TGF-β expression are also under study. Design studies on synthetic peptide antagonists to the TGF-β cell surface receptors have shown that linear peptide analogs of the amino acids 83-112 C-terminal binding region of the TGF-β ligand failed to bind to Tβ receptors, whereas the introduction of a disulfide bridge so as to constrain conformationally the peptides to a configuration similar to that of the native configuration of TGF-β1 and TGF-β2, yielded peptide binding to the non-signaling co-receptor TβREII and to the extracellular matrix protein/ligand trap decorin, which is known to bind to and inhibit activity of TGF-β. Thus, the constrained peptide may act on two signaling pathway steps to reduce TGF-β signaling. The peptides that were constrained conformationally failed to bind to the signaling Tβ RII receptor (Roswell Park Cancer Institute website, 2003).

Yet other small molecule inhibitors have been designed to block T receptors (Scios website, 2003) and small molecule (synthetic triterpenoids) enhancers of TGF-β signaling acting by increasing expression of Tβ type II receptor are also under study (Suh, et al., 2003). It was found that while the amount of TGF-β in extracellular fluid was increasing constantly due to increased secretion in diseased cells, the number of cells and receptors did not increase at a similar rate, and thus an inhibitor to a receptor may allow use of small amounts of therapeutic inhibitor agents.

Another approach is the use of a “soluble TβRII receptor” (actually a co-receptor) in which a chimeric IgG containing the extracellular portion of TβRII has been shown to control hepatic fibrosis in a rat liver injury model (George, et al., 1999), presumably by binding to and inhibiting TGF-β activity. Recombinant TβRII, the core protein/ligand trap (decorin) of a TGF-β binding proteoglycan and anti-TGF-β antibody have all been demonstrated to reduce lung fibrosis in a bleomycin-induced animal model (Wang, et al., 1999). The recombinant TβRII “soluble receptor” was thus proposed as a treatment for fibrotic diseases for which overexpression of TGF-β has been associated with excess collagen accumulation. U.S. Pat. No. 6,086,867, issued to the Whitehead Institute, discloses modulation of TGF-β by soluble Tβ type III (betaglycan co-receptor) polypeptides.

Another approach is to use a recombinant preparation of the ligand binding trap protein LAP (expressed in baculovirus), which has been demonstrated in vivo to inhibit activity of all three TGF-β isoforms and to inhibit activity of TGF-β1 in transgenic mice with elevated TGF-β1 levels (Bottinger, 1996). Antibody modulation of a novel protein (STRAP) that acts immediately downstream of the Tβ receptor has also been studied as a means of modulating TGF-β signaling (Vanderbilt University website, 2003). Use of anti-TGF-β2 monoclonal antibodies to prevent post-operative scarring after glaucoma surgery was the subject of a Phase II clinical trial co-sponsored by Genzyme and Cambridge Antibody Technologies (CAT). The use of anti-TGF-β1 monoclonal antibody as a treatment for diffuse scleroderma, was the subject of a Phase I/II clinical trial that was completed successfully by Genzyme and CAT. An initial clinical trial of the use of anti-TGF-β monoclonal antibodies as a treatment for idiopathic pulmonary fibrosis has been projected to begin in late 2003 by Genzyme and CAT.

U.S. Pat. No. 6,201,108, issued to the Whitehead Institute, discloses use of antibodies to Tβ type II and type III receptors for modulation of TGF-β activity. U.S. Pat. No. 5,662,904, issued to the Victoria University of Manchester, teaches anti-scarring by treatment of wounds with inhibitors to fibrotic growth factors such as TGF-β1 and TGF-β2, in which a wide range of inhibitors were used, e.g., antibodies to the growth factors, molecules binding the growth factor (e.g., decorin) to block ligand access to its receptors, antisense oligonucleotides and ribozymes binding growth factor mRNA and preventing its translation and soluble forms of the growth factor's receptors or binding domains of the receptors.

Yet another patent in this area includes U.S. Pat. No. 6,509,318, issued to the U. of California that discloses a family of hexapeptides, which are potent inhibitors of TGF-β1 activity. N- and C-terminal blocking groups were used to confer resistance to enzymatic degradation, and unnatural amino acids, e.g., N-methylalanine in place of alanine, were used to prevent premature renal clearance. Clinical use of the small peptides was cited as advantageous over antibodies and soluble receptors in that the antibodies and receptors are large proteins which are difficult to administer and deliver and can cause immune responses. In addition, the large amounts of TGF-β1 secreted in cancers require large amounts of neutralizing antibodies, exacerbating these problems. The approaches described hereinabove target either, TGF-β and TGF-β-related genes using antisense oligonucleotides or use proteins (antibodies and TGF fragments) targeted against different components of the TGF-β signaling pathway. These approaches, however, have failed to provide the specificity, half-life and lack of (or reduced) immunogenicity necessary for modulation of TGF-β signaling necessary to affect a wide variety of disease conditions.

### SUMMARY OF THE INVENTION

The present invention includes the selection and isolation of thioaptamers that target the signaling protein TGF-β1, compositions of such thioaptamers and the use of such thioaptamers to either block or enhance signal transduction of the TGF-β protein and thus function as, e.g., immunomodulatory agents. TGF-β is a multifunctional immunosuppressive cytokine, acting through both autocrine and paracrine mechanisms. TGF-β signaling controls a wide range of cell cycle processes including cell proliferation, recognition, differentiation and apoptosis. TGF-β is also involved in wound healing, metastasis, angiogenesis and immunosuppression.

In accordance with one embodiment of the present invention, the target molecule or portion thereof is TGF-β. In accordance with another embodiment of the present invention, the thioaptamer is selected to bind different isoforms of TGF-β or constituents thereof. In yet another embodiment of the present invention, the aptamer is selected to bind TGF-β, or constituents thereof, and wherein at least one nucleotide is an achiral thiophosphate or a dithiophosphate. In yet another embodiment of the present invention, the aptamer is selected to bind TGF-β, TGF-β receptors, co-receptors, downstream activators, enhancers or repressors and/or constituents thereof and wherein at least one nucleotide is an achiral thiophosphate or a dithiophosphate.

In yet another embodiment of the present invention, between 1 and 6 of the phosphate sites of the modified nucleotide aptamer are dithiophosphates. In another embodiment of the present invention, the modified nucleotide aptamer includes 6 to 12 dithioate linkages. In one embodiment of the invention, the thioaptamers further includes a detectable label. The detection method may include, e.g., colorimetric, chemiluminescent, fluorescent, radioactive, mass spectrometric, capacitance coupled electrical, Biacor or combinations thereof and the detectable label will be selected in accordance therewith. The present invention also includes aptamer and thioaptamer libraries containing multiple different but related members. In one embodiment of the present invention, the substrate for the library may be, e.g., beads, membranes, glass and combinations thereof. The substrate may even be a microarray of beads or other substrates.

The present invention includes a partially thio-modified aptamer (or thioaptamer) that binds to a TGF-beta protein, e.g., a human TGF-beta, which may be a TGF-beta dimer, a homodimer, a TGF-beta homodimer of TGF-beta 1, 2 or 3 homodimer of even a TGFbeta 1, 2 or 3 heterodimer. The partially thio-modified aptamer may include one or more thio-modifications as set forth in SEQ ID NOS: 4-22, and may be achiral. The thioaptamer may also include, e.g., a detectable label. Examples of detectable labels include, e.g., colorimetric, fluorescent, radioactive and/or enzymatic labels or agents. For use in vivo, ex vivo or in vitro, the thioaptamer may be dispersed or even lyophilized in one or more pharmaceutically acceptable salts. When provided along with one or more pharmaceutically acceptable salts, one or more pharmaceutically acceptable diluents may also be provided.

In another embodiment, the present invention includes a partially thio-modified aptamer that binds to a TGF-beta receptor. The TGF-beta receptor may be a signaling receptor and/or a co-receptor. In one example, the TGF-beta signaling receptor may be a human TGF-beta signaling receptor. Examples of TGF-beta signaling receptor include: ThetaRI and/or a ThetaRII receptor and may target, e.g., the GS domain of a ThetaRI receptor. Another target for the thioaptamers of the present invention includes the co-receptor TGF-beta 3.

In yet another embodiment, the partially thio-modified aptamer binds to a ligand-receptor complex that includes a TGF-beta ligand and a receptor, e.g., a ThetaRI and a ThetaRII receptors. A partially thio-modified aptamer may also be provided that binds to a ligand binding trap that traps TGF-beta ligands, e.g., the ligand binding trap targeted may be one of more of the following proteins: decorin, latency-associated protein (LAP) or alpha-macroglobulin. The partially thio-modified aptamers of the present invention may also bind to an auxiliary protein that promotes binding of TGF-beta ligand to Theta signaling receptors, e.g., the auxiliary protein may be a SARA protein. Alternatively, the partially thio-modified aptamer may bind to a Smad protein, e.g., the Smad protein may be an R-Smad, a Co-Smad, an I-Smad or combinations thereof.

In one specific embodiment, the partially thio-modified aptamer binds to a TGF-beta protein complex and enhances TGF-beta activity, e.g., by binding to a binding site on the TGF-beta protein complex that includes a region of a ligand binding trap protein or even an inhibitory I-Smad. Conversely, the partially thio-modified aptamer may binds a TGF-beta protein complex and inhibits TGF-beta activity, e.g., a binding site on the TGF-beta protein complex that includes a region of an R-Smad or a Co-Smad. In this embodiment, the partially modified thioaptamer inhibits TGF-beta activity by binding to a TGF-beta ligand, a TGF-beta ligand-Theta receptor complex, a TGF-beta signaling receptor and co-receptor, to an R-Smad or a Co-Smad. Alternatively, the partially modified thioaptamer may modify TGF-beta activity by binding to a TGF-beta ligand, a TGF-beta ligand-Theta receptor complex, a TGF-beta signaling receptor and co-receptor, to an R-Smad or a Co-Smad.

According to one embodiment of the present invention, the partially-modified nucleotide aptamer (“thioaptamer”) may include one or more, but not all the backbone links as phosphoromonothioate or phosphorodithioate (“phosphorothioates”) and may be DNA or RNA. Examples of the modifications include: dATP(αS), dTTP(αS), dCTP(αS) and/or dGTP(αS), dATP(αS₂), dTTP(αS₂), dCTP(αS₂) and/or dGTP(αS₂), mixtures and combinations thereof in which a combination of the sequence and selected modifications bind specifically to TGF-β. When in the form of RNA, the RNA thioaptamer is modified accordingly in the backbone and the bases. In another embodiment of the present invention, no more than three adjacent phosphate sites of the modified nucleotide aptamer are replaced with phosphorothioate groups. In yet another embodiment of the present invention, at least a portion of non-adjacent dA, dC, dG, or dT phosphate sites of the modified nucleotide aptamer are replaced with phosphorothioate groups. In yet another embodiment of the present invention, all of the non-adjacent dA, dC, dG, or dT phosphate sites of the modified nucleotide aptamer are replaced with phosphorothioate groups. In yet another embodiment of the present invention, all of the non-adjacent dA, dC, dG, and dT phosphate sites of the modified nucleotide aptamer are replaced with phosphorothioate groups. In still another embodiment of the present invention, substantially all non-adjacent phosphate sites of the modified nucleotide aptamer are replaced with phosphorothioate groups.

TGF-β has proliferative and non-proliferative effects on cells. TGF-β enhances the proliferation of certain cell types, such as osteoblasts and Schwann cells of the peripheral nervous system. TGF-β inhibits the proliferation of several types of cells, including capillary endothelial cells and smooth muscle cells, either by blocking cell cycle progress in the G₁ phase or by stimulating apoptosis, and can also alter differentiation of cells. TGF-β down regulates integrin expression involved in endothelial cell migration and also induces plasminogen activator inhibitors, which inhibit a proteinase cascade needed for angiogenesis and metastasis. TGF-β induces normal cells to inhibit transformed cells. TGF-β inhibition of cell proliferation may act as a regulatory mechanism to check the regeneration of certain tissue and may play a role in the initiation of carcinogenesis.

Alteration of normal TGF-β function is associated with the pathogenesis of a wide range of proliferative and inflammatory diseases. Increased TGF-β activity is implicated in many pathological conditions, including: fibrosis, scarring and adhesion during wound healing; fibrotic diseases of the lung, liver and kidney; atherosclerosis and arteriosclerosis; certain cancers such as gliomas, colon cancer, prostate cancer, breast cancer, neurofibromas, lung cancer; angiopathy, vasculopathy, nephropathy; systemic sclerosis; viral infections accompanied by immune suppression (HIV, HCV, CMV); and immunological disorders and deficiencies (auto-immune diseases).

Human TGF-β exists in either of three isoforms, TGF-β1, TGF-β2, and TGF-β3. Binding of TGF-β to cell surface receptors (TβRI and TβRII signaling receptors and the TβRIII co-receptor) initiates intracellular signaling leading to changes in the activity of gene regulatory proteins called Smads, which results in transcriptional activation of genes encoding regulators of cell division and cell death.

The thioaptamers of the present invention may target, e.g., TGF-β1 that has 390 amino acids, TGF-β2 that has 414 amino acids and/or TGF-β3 that has 410 amino acids. The isoforms share 70% amino acid identity and are coded by three distinct genes (Salzman, et al., 2002). Studies in Drosophila indicate that the promoter regions of the genes encoding the TGF-β isoforms had little sequence similarity and that the transcription factor Sp1 stimulated both TGF-β1 and TGF-β3 but not TGF-β2 (Geiser, et al., 1993). Structural differences between the TGF-β1 and TGF-β2 have been demonstrated based on NMR and X-ray studies (Hinck, et al., 1996).

Early studies indicated that the TGF-β isoforms exerted the same effects on immune cells in vitro (Schluessner, et al., 1990). Later studies showed that whereas many cell types respond equivalently to the isoforms, certain cell types respond selectively and the isoforms have differential activity towards binding proteins and cell surface receptors. TGF-β1 is a more potent inhibitor of colorectal cancer growth than is TGF-β2 (Burmester, et al., 1993). Whereas at low concentrations, the isoforms stimulate CSF-induced human myelopoiesis, at high concentrations only TGF-β2 is stimulatory and both TGF-β1 and TGF-β3 are inhibitory (Salzman, et al., 2002). These results are consistent with the finding that TGF-β1 and TGF-β3 have much higher binding affinity to the TβRII receptor than does TGF-β2 (Qian, et al., 1996). The isoforms have also been shown to exhibit different binding affinities to the TβRI receptor and similar binding affinities to the TβRIII co-receptor (Boyd and Massague, 1989).

TGF-β2 has a 10-fold greater binding affinity for the binding protein (ligand trap) α2-macroglobulin than does TGF-β1 (Burmester, et al., 1993) and also binds with higher affinity to a glycosyl phosphatidylinositol (GPI)-linked binding protein that is expressed on the surface of vascular endothelial cells (Qian, et al., 1999). TGF-β1, but not TGF-β2, binds to endoglin, a cell surface protein abundant in endothelial cells (Qian, et al., 1999). At least three different functional domains of the TGF-β molecule have been shown, in these studies, to be modulators of TGF-β interaction with binding proteins-amino acids 40-68 domain modulating interaction with endoglin, amino acids 92-98 domain modulating interaction with GPI-linked binding protein and amino acids 4047 domain modulating interaction with α-2 macroglobulin.

The high specificity of the combinatorial selection process for thioaptamers and its ability to select thioaptamers specific to any target protein is demonstrated herein. The thioaptamers of the present invention target differentially, e.g., TGF-β1 relative to the other TGF-β isoforms. Specific isoform targeting allows in vitro and in vivo study of the potential differential effects of the TGF-β isoforms and the further development of isoform-specific therapeutic agents. The high specificity of the combinatorial selection process for thioaptamers also yields thioaptamers that target differentially the Tβ receptors, thereby allowing in vitro and in vivo study of the role of the different Tβ receptors in the TGF-β signaling pathway.

The thioaptamers of the present invention may also target the various TGF-β receptors and combinations thereof. The human receptor serine/threonine kinase family includes twelve members—seven type I and five type II receptors—all of which are dedicated to TGF-β signaling (Manning, et al., 2002). Both receptor types have approximately 500 amino acids organized sequentially into an N-terminal extracellular ligand binding domain, a transmembrane region and a C-terminal serine/threonine kinase domain.

The cognate TGF-β ligand initiates signaling by binding to and bringing together type I and type II receptor serine/threonine kinases on the cell surface, thus forming an “active receptor signaling complex,” which allows the type II receptor to phosphorylate the type I receptor kinase domain and which propagates the signal by directly phosphorylating the “receptor-regulated” R-Smad proteins (Smads 2, 3 involved in TGF-β subfamily signaling and Smads 1, 5 and 8 involved in BMP subfamily signaling). Once activated, the R-Smads undergo homotrimerization and form a heteromeric complex with the Co-Smad (Smad 4). The R-Smad-Co-Smad complex translocates to the nucleus of the cell, and in conjunction with other nuclear factors, regulates the transcription of genes. The inhibitory I-Smads (Smads 6, 7) may regulate negatively TGF-β signaling by competing with R-Smads for either receptor or Co-Smad interaction and/or by targeting the receptors for ubiquitination and degradation. The aptamers and thioaptamers of the present invention may be used alone or in combination, e.g., by providing two or more thioaptamers that are specific for the TGF-β ligand and/or its receptors and/or inhibitors to provide combination thioaptamer therapy.

The active form of the TGF-β ligand is a dimer, stabilized by hydrophobic interactions, which in most cases are further strengthened by an intersubunit disulfide bridge. Each monomer includes several extended beta strands interlocked by conserved disulfide bonds to form a tight structure called a “cysteine knot” (Sun and Davies, 1995). The dimeric arrangement of the TGF-β ligand suggests formation of a complex with two type I and two type II receptors, with ligand access to the receptors regulated by a family of soluble proteins known as ligand binding traps, which can block the ligand surfaces required to bind to the receptors. Ligand traps of TGF-β include the proteoglycan decorin, the circulating protein α-2 macroglobulin and the proregion of the TGF-β precursor known as “latency-associated polypeptide” or LAP (Bottinger, et al., 1996; Shi, et al., 2003). Thioaptamers that bind selectively to specific ligand trap proteins may be used to study the role of the different ligand traps in regulating TGF-β signaling and the effects of short-circuiting the regulatory role of such ligand traps (thus enhancing TGF-β signaling).

TGF-β ligands exhibit high affinity for the type II receptors and do not interact with isolated type I receptors. Thus, the TGF-β ligand first binds tightly to the ectodomain of the type II receptor and following that, the type I receptor is incorporated into a ligand-receptor complex that includes a TGF-β ligand dimer and four receptor molecules. Structural analysis of the complex between TGF-β3 and TβRII receptor indicates that binding occurs at the far ends (“fingertips”) of the elongated TGF-β ligand dimer (Hart, et al., 2002). Each receptor binds to one monomer of the β3 dimer, creating two symmetrically positioned concave surfaces, postulated to act as the binding sites for the type 1 receptor. The present invention includes the selection of thioaptamers that bind to different regions of the TGF-β ligand-receptor complex that either reduce or increase TOF-β signaling.

Two alternative models have been proposed to explain how binding of the TGF-β ligand to its type II receptor induces incorporation of the type I receptor into the complex. In one model, the large conformational change in the ligand due to binding to its type II receptor exposes the ligand's binding epitope to the type I receptor. In the other model, the type I receptor interacts with the extended surface of the ligand-type II receptor complex (Shi, et al., 2003). The present invention is not constrained or limited in any way by or to any such model.

The type I receptor, but not the type II, contains a characteristic SGSGSG sequence, the “GS domain,” immediately N-terminal to the kinase domain. The type II receptor phosphorylates multiple serine and threonine residues of the type I GS domain, thereby activating type I. Thus, the GS domain of the type I receptor serves as an important regulatory domain for TGF-beta signaling. As such, the GS domain of the type I receptor is a candidate for thioaptamer targeting, to facilitate study of its role in TGF-β signaling. For example, the immunophilin FKBP12 was shown to inhibit TGF-β signaling by binding to the unphosphorylated GS domain of type I receptor (Huse, et al., 1999), so that it cannot interact with its downstream target, the R-Smad proteins.

Access of TGF-β ligands to their receptors is controlled by two classes of molecules, the ligand binding traps and the co-receptors. The ligand binding traps are soluble proteins that bind to the TGF-β ligand and bar its access to membrane receptors. The ligand trap protein decorin binds strongly to TGF-β (Yamaguchi, et al., 1990). In another embodiment of the present invention, thioaptamers may be targeted against the ligand trap proteins to modulate TGF-β signaling pathway. The co-receptors are membrane-anchored proteins that promote ligand binding to the signaling receptors. For example, betaglycan, also known as the TGF-β type III receptor, mediates TGF-β binding to the type H receptor, a role which is critical for TGF-β2 signaling (Massague, et al., 1998). Thioaptamers may also be targeted alone or in combination with other thioaptamers against the ligand, the receptors, the ligand trap protein(s) and/or the co-receptors to modulate TGF-β signaling pathway.

The recognition of R-Smads by the TGF-β receptors is facilitated by auxiliary proteins. For example, the R-Smads 2 and 3 can be immobilized specifically near the cell surface by the “Smad anchor for receptor activation,” or SARA protein (T. Tsukazaki, et al., 1998) through interactions between the SARA sequence and an extended hydrophobic surface area on the Smad (G. Wu, et al., 2000). The interactions allow for more efficient recruitment of the Smads to the receptors for phosphorylation. The SARA protein is thus another viable thioaptamer target for study of the TGF-β signaling pathway.

Many somatic and hereditary disorders result from mutations or malfunctions in the TGF-β pathway (Massague, et al., 2000). For example, inactivating mutations in the TβRII receptor gene occur in most human colorectal and gastric carcinomas (Markowitz, et al., 1995), including hereditary nonpolyposis colon cancer (Akiyama, et al., 1997) and inactivating mutations in the TβRI receptor have been detected in a third of ovarian cancers (Wang, et al., 2000) and in metastatic breast cancers (Chen, et al., 1998). Deletions in the gene for the TβRI receptor have been found at low frequency in pancreatic cancers (Goggins, et al., 1998) and in cutaneous T-cell lymphoma (Schiemann, et al., 1999).

The high binding affinity and selectivity of partially thioated thioaptamers and their nuclease resistance may be used to reduce or enhance TGF-β signaling by selection of thioaptamers binding to a variety of targets—(1) the TGF-β ligand itself (or to a specific isoform of the TGF-β ligand); (2) the ligand-receptor complex; (3) either of the Tβ receptors (type I or type II), including specific regions of a receptor such as the GS domain of the type I receptor; (4) either of the ligand traps known to regulate ligand access to the receptors; (5) co-receptors known to enhance ligand-receptor binding, such as the Tβ type III receptor, betaglycan; (6) auxiliary proteins such as the SARA protein, which enhance R-Smad activation by receptors; (7) R-Smads; (8) Co-Smads; (9) R-Smad-Co-Smad complexes; and/or (10) I-Smads. Enhancement of TGF-β signaling may be possible through binding of selective thioaptamers to the ligand-receptor complex, to ligand traps or to I-Smads. The thioaptamers found to be most effective in modulating TGF-β signaling can then be tested on in vivo animal models and candidates then selected for potential use as therapeutic agents in diseases involving TGF-β over-expression or under-expression.

The present invention also includes a method of inhibiting TGF-β activity that includes the step of providing to a host in need of therapy a pharmaceutically effective amount of a thioaptamer that specifically binds to and inhibits TGF-β activity. The thioaptamer is provided to a host in order to ameliorate the effects of, e.g., fibrosis, scarring and adhesion during wound healing; fibrotic diseases of the lung, liver and kidney; atherosclerosis, arteriosclerosis; cancers including gliomas, colon cancer, prostate cancer, breast cancer, neurofibromas, lung cancer; angiopathy, vasculopathy, nephropathy; systemic sclerosis; viral infections accompanied by immune suppression (HIV, HCV); and immunological disorders and deficiencies (auto-immune diseases).

The present invention also includes a method of quantitating TGF-β levels in a sample by contacting a sample with a TGF-β-specific thioaptamer. In this embodiment, the sample may be, e.g., a physiological sample, i.e., blood, tissue, cells (e.g., cells from a biopsy, lavage, swab), supernatant or media in which cells have been dispersed and/or cultures. To modulate TGF-β signaling the user may administer to a host a TGF-β specific thioaptamer that modulates the activity through the TGF-β receptor in a dosage effective to reduce activity of the TGF-β. Modification of TGF-β signaling includes, e.g., increasing or decreasing activity, depending on the effect desired. Up or down regulation may be by providing a cell, tissue or host with a thioaptamer selected from the group consisting of SEQ ID NOS.: 4-22. As such, the present invention includes a method of treating a pathological condition due to increased TGF-β activity that includes the steps of, e.g., administering to a host an effective dosage of a thioaptamer that modulates TGF-β, e.g., the thioaptamer binds to TGF-β, the TGF-β receptor, a TGF-β auxiliary protein, a TGF-β ligand binding trap protein and/or a Smad protein. The thioaptamer may modulate activity through the TGF-β, receptor by increasing or decreasing activity.

The thioaptamer of the present invention may be provided through a variety of routes, e.g., ip, iv, sc, im, orally, etc. For some uses, the TGF-β specific thioaptamer may be, e.g., encapsulated, powdered, in aqueous or other solution, tableted, etc. When provided in a capsule, e.g., the capsule may be degradable by an external stimulus to release the TGF-β specific thioaptamer, e.g., by exposing the capsule to, e.g., UV light, acid, water, in vivo enzymes, ultrasound and/or heat. The TGF-β specific thioaptamer may also be bound, covalently or non-covalently a binding molecule. When the TGF-β, specific thioaptamer is bound to a binding molecule the binding molecule may be detached by one or more of the external stimuli described hereinabove to provide, e.g., a host with the TGF-β, specific thioaptamer at a specific location.

As such, the thioaptamer of the present invention may also be used in a method of treating a pathological condition in which increased TGF-β activity has been implicated by administering to a host a TGF-β specific thioaptamer in a pharmaceutically acceptable carrier at a dosage effective to reduce TGF-β activity. The pharmaceutically acceptable carrier may be, e.g., a cream, gel, aerosol and powder for internal or external application, e.g., topical application. The pharmaceutically acceptable carrier may be, e.g., a sterile solution for injection, irrigation and even inhalation.

To reduce scarring, e.g., the thioaptamer may be provided in a pharmaceutically acceptable carrier that includes a sterile dressing for topically covering a wound. Examples of pharmaceutically acceptable carriers include biocompatible: biopolymers and/or biodegradable polymers for implanting within or about a wound. In some specific embodiments, the thioaptamer may be provided along with one or more growth factors. The thioaptamer may also be used in a method of modulating TGF-β signaling by administering to a host a TGF-β ligand binding trap specific thioaptamer that modulates the activity through the TGF-β receptor in a dosage effective to reduce activity of the TGF-β. The method of modulating TGF-β signaling may be by administering to a host a TGF-β auxiliary protein specific thioaptamer that modulates the activity through the TGF-β receptor in a dosage effective to reduce activity of the TGF-β or even a method of modulating TGF-β3 signaling by administering to a host a TGF-β Smad protein specific thioaptamer that modulates the activity through the TGF-β3 receptor in a dosage effective to reduce activity of the TGF-β.

### BRIEF DESCRIPTION OF THE DRAWINGS

For a more complete understanding of the features and advantages of the present invention, reference is now made to the detailed description of the invention along with the accompanying figures and in which:

FIG. 1 is a Clustal W (1.8) alignment of sequences of clones isolated during the selection of thioaptamer binding TGF-β1 (through 12′ round of selection);

FIG. 2 is a predicted secondary structures of highest affinity (to TGF-β1) thioapter clones of rounds 5,9,12 (thioaptamers T5_{—}14, T9_{—}5, T12_{—}8);

FIGS. 3a, 3b, 3c and 3d are gels of electromobility shift assays of the initial library (3a) and of thioaptamer candidates T5_{—}14, T9_{—}5 and T9_{—}22 (3b, 3c, 3d);

FIGS. 4a, 4b and 4c show models of a predicted molecular nature of three DNA bands in the corresponding portions of a gel; and

FIG. 5 is an analysis of binding of T9_{—}5, T9_{—}22 and initial library to a TGF-β1 target protein.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, “synthesizing” of a random combinatorial library refers to chemical methods known in the art of generating a desired sequence of nucleotides including where the desired sequence is random. Typically in the art, such sequences are produced in automated DNA synthesizers programmed to the desired sequence. Such programming can include combinations of defined sequences and random nucleotides.

“Random combinatorial oligonucleotide library” is used to describe a large number of oligonucleotides of different sequence where the insertion of a given base at given place in the sequence is random. “PCR primer nucleotide sequence” refers to a defined sequence of nucleotides forming an oligonucleotide which is used to anneal to a homologous or closely related sequence in order form the double strand required to initiate elongation using a polymerase enzyme. “Amplifying” means duplicating a sequence one or more times. Relative to a library, amplifying refers to en masse duplication of at least a majority of individual members of the library.

As used herein, “thiophosphate” or “phosphorothioate” are used interchangeably to refer analogues of DNA or RNA having sulphur in place of one or more of the non bridging oxygens bound to the phosphorus. Monothiophosphates or phosphoromonothioates [αS] have only one sulfur and are thus chiral around the phosphorus center. Dithiophosphates are substituted at both oxygens and are thus achiral. Phosphoromonothioate nucleotides are commercially available or can be synthesized by several different methods known in the art. Chemistry for synthesis of the phosphorodithioates has been developed by one of the present inventors as set forth in U.S. Pat. No. 5,218,088, issued to Gorenstein, D. G. and Farschtschi, N., issued Jun. 8, 1993, for a Process for Preparing Dithiophosphate Oligonucleotide Analogs via Nucleoside Thiophosphoramidite Intermediates, relevant portions incorporated herein by reference.

When discussing changes to oligonucleotides, “Modified” is used herein to describe oligonucleotides or libraries in which one or more of the four constituent nucleotide bases of an oligonucleotide are analogues or esters of nucleotides normally comprising DNA or RNA backbones and wherein such modification confers increased nuclease resistance. Thiophosphate nucleotides are an example of modified nucleotides. “Phosphodiester oligonucleotide” means a chemically normal (unmodified) RNA or DNA oligonucleotide. Amplifying “enzymatically” refers to duplication of the oligonucleotide using a nucleotide polymerase enzyme such as DNA or RNA polymerase. Where amplification employs repetitive cycles of duplication such as using the “polymerase chain reaction”, the polymerase may be, e.g., a heat stable polymerase, e.g., of Thermus aquaticus or other such polymerases, whether heat stable or not. When discussing the effect of an thioaptamer on TGF-β signaling, the term “modified” is used to describe a change in the activity of the TGF-β receptor, receptor complex or related proteins that may upregulate or downregulate the activity of host TGF-β activity, including, e.g., actual signaling via second messengers, up or down regulation of gene expression, message translation, message retention, siRNA induction or gene product production or even degradation.

“Contacting” in the context of target selection means incubating a oligonucleotide library with target molecules. “Target molecule” means any molecule to which specific aptamer selection is desired. “Target protein” means any peptide or protein molecule to which a specific aptamer selection is desired. “Essentially homologous” means containing at least either the identified sequence or the identified sequence with one nucleotide substitution. “Isolating” in the context of target selection means separation of oligonucleotide/target complexes, preferably DNA/protein complexes, under conditions in which weak binding oligonucleotides are eliminated.

By “split synthesis” it is meant that each unique member of the combinatorial library is attached to a separate support bead on a two (or more) column DNA synthesizer, a different thiophosphoramidite or phosphoramidite is first added onto both identical supports (at the appropriate sequence position) on each column. After the normal cycle of oxidation (or sulfurization) and blocking (which introduces the phosphate, monothiophosphate or dithiophosphate linkage at this position), the support beads are removed from the columns, mixed together and the mixture reintroduced into both columns. Synthesis may proceed with further iterations of mixing or with distinct nucleotide addition.

Aptamers may be defined as nucleic acid molecules that have been selected from random or unmodified oligonucleotides (“ODN”) libraries by their ability to bind to specific targets or “ligands.” An iterative process of in vitro selection may be used to enrich the library for species with high affinity to the target. The iterative process involves repetitive cycles of incubation of the library with a desired target, separation of free oligonucleotides from those bound to the target and amplification of the bound ODN subset using the polymerase chain reaction (“PCR”). The penultimate result is a sub-population of sequences having high affinity for the target. The sub-population may then be subcloned to sample and preserve the selected DNA sequences. These “lead compounds” are studied in further detail to elucidate the mechanism of interaction with the target.

“Detectable labels” are compounds and/or elements that can be detected due to their specific functional properties and/or chemical characteristics, the use of which allows the agent to which they are attached to be detected, and/or further quantified if desired, such as, e.g., an enzyme, an antibody, a linker, a radioisotope, an electron dense particle, a magnetic particle and/or a chromophore or combinations thereof, e.g., fluorescence resonance energy transfer (FRET). There are many types of detectable labels, including fluorescent labels, which are easily handled, inexpensive and nontoxic.

The present inventors recognized that it is not possible to simply substitute thiophosphates in a sequence that was selected for binding with a normal phosphate ester backbone oligonucleotide. Simple substitution was not practicable because the thiophosphates can significantly decrease (or increase) the specificity and/or affinity of the selected ligand for the target. It was also recognized that thiosubstitution leads to a dramatic change in the structure of the aptamer and hence alters its overall binding affinity.

The present invention takes advantage of the “stickiness” of thio- and dithio-phosphate ODN agents to enhance the affinity and specificity to a target molecule. In a significant improvement over existing technology, the method of selection concurrently controls and optimizes the total number of thiolated phosphates to decrease non-specific binding to non-target proteins and to enhance only the specific favorable interactions with the target. The present invention permits control over phosphates that are to be thio-substituted in a specific DNA sequence, thereby permitting the selective development of aptamers that have the combined attributes of affinity, specificity and nuclease resistance.

In one embodiment of the present invention, a method of post-selection aptamer modification is provided in which the therapeutic potential of the aptamer is improved by selective substitution of modified nucleotides into the aptamer oligonucleotide sequence. An isolated and purified target binding aptamer is identified and the nucleotide base sequence determined. Modified achiral nucleotides are substituted for one or more selected nucleotides in the sequence. In one embodiment, the substitution is obtained by chemical synthesis using dithiophosphate nucleotides. The resulting aptamers have the same nucleotide base sequence as the original aptamer but, by virtue of the inclusion of modified nucleotides into selected locations in the sequences, improved nuclease resistance and affinity is obtained.

RNA and DNA oligonucleotides (ODNs) can act as “aptamers,” (i.e., as direct in vivo inhibitors selected from combinatorial libraries) for a number of proteins, including viral proteins such as HIV RT and transcription factors such as, e.g., human NF-κB, AP-1, NF IL-6 or other proteins involved in, e.g., transcription. Decoy ODNs were developed to inhibit expression from CRE and AP-1 directed transcription in vivo and inhibit growth of cancer cells in vitro and in vivo. Yet others have demonstrated the potential of using specific decoy and aptamer ODNs to bind to various proteins, serve as therapeutic or diagnostic reagents, and to dissect the specific role of particular transcription factors in regulating the expression of various genes. In contrast to antisense agents, duplex aptamers appear to exhibit few if any non-specific effects.

Among a large variety of modifications, S-ODN and S₂-ODN render the agents more nuclease resistant. The first antisense therapeutic drug uses a modified S-ODN. The S₂-ODNs also show significant promise, however, the effect of substitution of more nuclease-resistant thiophosphates cannot be predicted, since the sulfur substitution can lead to significantly decreased (or increased) binding to a specific protein as well as structural perturbations and thus it is not possible to predict the effect of backbone substitution on an aptamer selected combinatorially. Hence, the present inventors recognized that selection should be carried out simultaneously for both phosphate ester backbone substitution and base sequence.

Phosphorodithioate analogs have been synthesized to produce an important class of sulfur-containing oligonucleotides, the dithiophosphate S₂-ODNs. These dithioates include an internucleotide phosphodiester group with sulfur substituted for both non-linking phosphoryl oxygens, so they are both isosteric and isopolar with the normal phosphodiester link, and are also highly nuclease resistant. One group showed highly effective protection of the dithioate against degradation by endogenous nucleases after 58% backbone modification. Significantly, the S₂-ODNs, in contrast to the phosphoramidite-synthesized monothiophosphate (S-ODNs), are achiral about the dithiophosphate center, so problems associated with diastereomeric mixtures are completely avoided. The S₂-ODNs and the DNs, are taken up efficiently by cells, especially if encapsulated in liposomes.

Thiophosphate aptamers are capable of specifically and non-specifically binding to proteins. Importantly, the present inventors have observed that sulfurization of the phosphoryl oxygens of oligonucleotides often leads to their enhanced binding to numerous proteins. The dithioate agents, for instance, appear to inhibit viral polymerases at much lower concentrations than do the monothiophosphates, which in turn are better than the normal phosphates, with K_{d}'s for single strand aptamers in the nM to sub-nM range for HIV-1 RT and NF-κB. For HIV-1 RT, dithioates bind 28-600 times more tightly than the normal aptamer oligonucleotide or the S-analogue. Sequence is also important, as demonstrated by the observation that a 14-nt dithioate based on the 3′ terminal end of human tRNA^{Lys} complementary to the HIV primer binding site is a more effective inhibitor (ID₅₀=4.3 nM) than simply dithioate dC₁₄ (ID₅₀=62 nM) by an order of magnitude.

Oligonucleotides with high monothio- or dithiophosphate backbone substitutions appear to be “stickier” towards proteins than normal phosphate esters, an effect often attributed to “non-specific interactions.” One explanation for the higher affinity of the thiosubstituted DNAs is the poor cation coordination of the polyanionic backbone sulfur, being a soft anion, does not coordinate as well to hard cations like Na⁺, unlike the hard phosphate oxyanion. The thiosubstituted phosphate esters then act as “bare” anions, and since energy is not required to strip the cations from the backbone, these agents appear to bind even more tightly to proteins.

As used herein, the terms “thio-modified aptamer,” “thioaptamer” and/or “partially thio-modified aptamer” are used interchangeably to describe oligonucleotides (ODNs) (or libraries of thioaptamers) in which one or more of the four constituent nucleotide bases of an oligonucleotide are analogues or esters of nucleotides that normally form the DNA or RNA backbones and wherein such modification confers increased nuclease resistance; and the DNA or RNA may be single or double stranded. For example, the modified nucleotide thioaptamer can include one or more monophosphorothioate or phosphordithioate linkages selected by incorporation of modified backbone phosphates through polymerases from wherein the group: dATP(αS), dTTP(αS), dCTP(αS), dGTP(αS), rUTP (αS), rATP(αS), rCTP(αS), rGTP(αS), dATP(αS₂), dTTP(αS₂), dCTP(αS₂), dGTP(αS₂), rATP(αS₂), rCTP(αS₂), rGTP(αS₂) and rUTP(αS₂) or modifications or mixtures thereof. Phosphoromonothioate or phosphorodithioate linkages may also be incorporated by chemical synthesis or by DNA or RNA synthesis by a polymerase, e.g., a DNA or an RNA polymerase or even a reverse transcriptase, or even thermostable or other mutant versions thereof. In another example, no more than three adjacent phosphate sites of the modified nucleotide aptamer are replaced with phosphorothioate groups. In yet another example, at least a portion of non-adjacent dA, dC, dG, dU or dT phosphate sites of the modified nucleotide aptamer are replaced with phosphorothioate groups. In another example of a thioaptamer, all of the non-adjacent dA, dC, dG, or dT phosphate sites of the modified nucleotide aptamer are replaced with phosphorothioate groups; all of the non-adjacent dA, dC, dG, and dT phosphate sites of the modified nucleotide aptamer are replaced with phosphorothioate groups; or substantially all non-adjacent phosphate sites of the modified nucleotide aptamer are replaced with phosphorothioate groups. In still another embodiment of the present invention, no more than three adjacent phosphate sites of the modified nucleotide aptamer are replaced with phosphorodithioate groups. The thioaptamers may be obtained by adding bases enzymatically using a mix of four nucleotides, wherein one or more of the nucleotides are a mix of unmodified and thiophosphate-modified nucleotides, to form a partially thiophosphate-modified thioaptamer library. In another example of “thioaptamers” these are made by adding bases to an oligonucleotide wherein a portion of the phosphate groups are thiophosphate-modified nucleotides, and where no more than three of the four different nucleotides are substituted on the 5′-phosphate positions by 5′-thiophosphates in each synthesized oligonucleotide are thiophosphate-modified nucleotides.

Thioaptamers and other nucleic acid analogs (e.g., peptide nucleic acids (PNAs), methylphosphonates, etc.) are emerging as important agents in therapeutics, drug discovery and diagnostics. Three key attributes define the unique ability of (thio)aptamers to perform their essential functions: (1) they target specific proteins in physiological pathways; (2) their sequence and structure is not intuitively obvious from canonical biologics and oftentimes can only be deduced by combinatorial selection against their targets; and (3) they bind their targets with higher affinities than do naturally occurring nucleic acid substrates. Importantly, the backbone modifications of thioaptamers and their nucleic acid backbone analogs enable aptamers to be introduced directly into living systems with in vivo lifetimes many times greater than unmodified nucleic acids, due to their inherent nuclease resistance of the modified aptamers. The inherent nuclease resistance is extraordinarily important for their efficacy in use.

In vitro combinatorial selection of thiophosphate aptamers may be used with the present invention. A recent advance in combinatorial chemistry has been the ability to construct and screen large random sequence nucleic acid libraries for affinity to proteins or other targets. The aptamer and/or thioaptamer nucleic acid libraries are usually selected by incubating the target (protein, nucleic acid or small molecule) with the library and then separating the non-binding species from the bound. The bound fractions may then be amplified using the polymerase chain reaction (PCR) and subsequently reincubated with the target in a second round of screening. These iterations are repeated until the library is enhanced for sequences with high affinity for the target. However, agents selected from combinatorial RNA and DNA libraries have previously always had normal phosphate ester backbones, and so would generally be unsuitable as drugs or diagnostics agents that are exposed to serum or cell supernatants because of their nuclease susceptibility. The effect of substitution of nuclease-resistant thiophosphates cannot be predicted, since the sulfur substitution can lead to significantly decreased (or increased) binding to a specific protein.

The present inventors have described the combinatorial selection of phosphorothioate oligonucleotide aptamers from random or high-sequence-diversity libraries, based on tight binding to the target (e.g. a protein or nucleic acid) of interest, U.S. patent application Ser. No. 10/120,815, relevant portions incorporated herein by reference. An in vitro selection approach for RNA thioaptamers has also been described Ellington and co-workers.

One approach used by the inventors is a hybrid monothiophosphate backbone. Competition assay for binding 42-mer aptamers (ODN) were conducted. In standard competitive binding assays, ³²P-IgkB promoter element ODN duplex was incubated with recombinant p50 or p65 and competitor oligonucleotide. The reactions were then run on a nondenaturing polyacrylamide gel, and the amount of radioactivity bound to protein and shifted in the gel was quantitated by direct counting.

In another example a combinatorial library was created by PCR, using an appropriate dNTP(αS) in the Taq polymerization step. A combinatorial thiophosphate duplex and single stranded (ss) libraries was screened successfully for binding to a number of different protein and nucleic acid targets, including: TGF-α, NF-IL6, NF-κB, HIV reverse transcriptase, Venezuelan Equine Encephalitis nucleocapsid (using an RNA thioaptamer), HepC IRES nucleic acid, and others, including a protein involved in CpG-induced “innate immunity.”

Briefly, a filter binding method was used that was modified to minimize non-specific binding of the DNs to the nitrocellulose filters. A column method may also be used in which the target is covalently attached to a column support for separation as well. The duplex, ssDNA and/or ssRNA DN's are eluted from the filter under high salt and protein denaturing conditions. Subsequent ethanol precipitation and for the duplex DNA DNs, another Taq polymerase PCR thiophosphate amplification provided product pools for additional rounds of selection (for RNA thioaptamers RT and T7 polymerase were used). To increase the binding stringency of the remaining pool of DNs in the library and select higher-affinity members, the KCl concentration was increased and the amount of protein in subsequent rounds was reduced as the iteration number increased. After cloning, the remaining members of the library were sequenced, which allowed for “thioselect”™ simultaneously for both higher affinity and more nuclease-resistant, “thioaptamer™” agents. For example, the thioselection method has been used to isolate a tight-binding thioaptamer for 7 of 7 targets tested.

TGF-β in wound healing (major function of TGF-β1). Wound healing in tissue involves a process of extracellular matrix biosynthesis, turnover and organization, which commonly leads to the production of fibrous connective tissue scars and consequential loss of normal tissue function. The extracellular matrix biosynthesis process is regulated by a number of soluble growth factors secreted within the wound environment (especially by platelets and macrophages). These soluble growth factors include TGF-α, TGF-β (isoforms β1, β2, and β3), platelet-derived growth factor PDGF, epidermal growth factor EGF, the insulin growth factors IGF-I and IGF-III, and acidic and basic fibroblast growth factors FGF. TGF-β is secreted in excessive amounts in wounds and plays a major role in directing cellular response to injury, driving fibrogenesis and potentially underlying the progression of chronic injury to fibrosis (George, et al., 1999). It has been proposed both that wound healing can be promoted by administering such growth factors (Sporn, et al., 1983) and that scarring can be reduced without compromising wound healing by administering inhibitors to fibrotic growth factors such as PDGF and TGF-β1 and TGF-β2 (Shah, et al., 1992; U.S. Pat. No. 5,662,904, U.S. Pat. No. 5,683,988). The inventors of U.S. Pat. No. 5,662,904 teach that TGF-β3 is not a fibrotic growth factor and that its inhibition is counter-productive in that scarring is not reduced, whereas would healing might be slowed. Wound healing thus provides an opportunity for thioaptamer development, e.g., thioaptamers that bind to TGF-β1 and/or TGF-β2 are selected that do not bind to TGF-β3, or which inhibit steps in the signaling pathway of TGF-β1 and TGF-β2 without inhibiting the signaling pathway of TGF-β3.

TGF-β and cancer. The present invention may be used to target specifically isoforms of TGF-β in certain cancers, in which overexpression of TGF-β makes tumor cells more invasive and metastatic by increasing angiogenesis, suppressing the immune system and altering the interactions of tumor cells with the extracellular matrix (Gorelik and Flavell, 2002). For example, TGF-β is overexpressed in malignant gliomas and in the most common forms of cancer, including colon cancer, breast cancer and prostate cancer. TGF-β1 overexpression has also been demonstrated in prostate cancer (Truong, 1993), colon cancer (Anzano, 1989; Tsushima, et al., 1996; Robson, et al., 1996; Zanetti, et al., 2003; Nagayama, et al., 2002), liver cancer (Ito, et al., 1995), non-small cell lung cancer (Hasegawa, et al., 2001) and breast cancer (Kong, 1995).

The thioaptamers of the present invention may also be used to target TGF-β2, which is overexpressed by human glioma cells, such as gliobastomas (Grimm, 1988). Importantly, there is currently no satisfactory treatment for human glioma tumors. TGF-β immunosuppressive effects reduce the proliferation of cytotoxic T-lymphocytes that otherwise could be able to destroy the glioma cells. TGF-β's inhibitory effect on T-cell proliferation is attributed to inhibition of 1L-2 production (Brabletz, 1993), which inhibits the proliferation of normal T-lymphocytes (Grimm, et al., 1988; Kuppner, et al., 1989; Sawamura, et al., 1990) and to inhibition of T-cell differentiation (Swain, 1991).

Overexpression of all three TGF-β isoforms has been demonstrated in breast cancer (MacCallum, 1994; Vrana, et al., 1996), and it has been hypothesized that tumor-derived immunosuppressive TGF-β is responsible for the poor efficacy observed in initial clinical trials of dendritic cell-based antitumor vaccines and that a means of inhibiting TGF-β activity must be combined with such vaccines (Kao, et al., 2003). The present invention may be used as a single or combination therapy, including highly specific thioaptamers targeting any number of members of the TGF-β signaling pathway, to target and control the effects of one or more of the TGF-β isoforms.

TGF-β and viral infections. TGF-β1 has also been shown to promote the depletion of CD4⁺ T-cells after HIV-1 infection, by inducing apoptosis, possibly contributing to pathogenesis in vivo (Wang, et al., 2001). For example, 25% of HIV-positive donors were found to produce TGF-β1 in response to stimulation with HIV proteins, with the TGF-β1 production sufficient to significantly depress the IFN-γ response of CD8⁺ T-cells to HIV proteins. The suppression was reversed by anti-TGF-β1 antibody. TGF-β1 production by HIV-infected CD8⁺ T-cells may represent an important mechanism by which an HIV-specific response can nonspecifically suppress HIV-specific immune responses (Garda, et al., 2002). As such, the present invention may be used to target TGF-β1 to prevent CD8+ suppression and even lack of macrophage activation. In vitro studies of primary macrophages infected with HIV-1 demonstrated that viral replication was preceded by increased secretion of TGF-β1, and was partially reversed by anti-TGF-β1 antibody. Positive correlation was observed between TGF-β1 production and HIV-1 growth (Lima, et al., 2002). The HIV envelope protein, gp160, a superantigen being tested in several HIV vaccine trials, has been shown to up-regulate TGF-β1 in mucosal, tonsil-originating B cells (Cognasse, et al., 2003).

Research on CMV encephalitis has shown that infected astrocytes induce TGF-β production, which in turn enhances CMV expression. Astrocyte release of CMV was reduced by anti-TGF-β antibody, therefore, the thioaptamers of the present invention may be used to reduce TGF-β activity during viral infection with, e.g., CMV. Astrocyte release of CMV was increased significantly after addition of exogenous TGF-β (Kossman, et al., 2003). HCV proteins have been shown to alter signal transduction in infected hepatocytes, inducing the production of profibrogenic mediators, in particularly TGF-β, leading to proposed use of TGF-β inhibitors for treatment of HCV (Schuppan, et al., 2003).

Methods for modulation of TGF-β activity. The involvement of TGF-β in the biological pathways of a host of diseases and in wound healing has led to extensive research for both inhibiting and activating TGF-β. The thioaptamers of the present invention may also be used as part of a sole or combination therapy for modulation of, e.g., TGF-β activity, including antisense oligonucleotides binding to one or more of the TGF-β isoforms, Tβ receptors, soluble Tβ receptors, and the ligand binding traps decorin and LAP.

### EXAMPLE 1

S-ODN, S₂-ODN and monothio-RNA Split and Pool Synthesis. A split and pool synthesis combinatorial chemistry method was developed for creating combinatorial S-ODN, S₂-ODN and monothio-RNA libraries (and readily extended to unmodified ODNs-whether single strand or duplex). In this procedure each unique member of the combinatorial library was attached to a separate support bead. Targets that bind tightly to only a few of the potentially millions of different support beads can be selected by binding the targets to the beads and then identifying which beads have bound target by staining and imaging techniques. The methodology of the present invention allowed the rapid screening and identification of thioaptamers that bind to proteins such as NF-κB using a novel PCR-based identification tag of the selected bead.

The dA, dG, dC and dT phosphoramidites were purchased from Applied Biosystems (Palo Alto, Calif.) or Glen Research (Sterling, Va.). The Beaucage reagent (3H-1,2-Benzodithiol-3-one 1,1-dioxide) was from Glen Research. The Taq polymerase kits were from Applied Biosystems. The TA Cloning kit was from Invitrogen. The Klenow DNA polymerase I was from Promega. Polystyrene beads (60-70 μm) with non-cleavable hexaethyleneglycol linkers with a loading of 36 μmol/g were from ChemGenes Corp (Ashland, Mass.). The Alexa Fluor 488 dye was from Molecular Probes, Inc (Eugene, Oreg.). The dA, dG, dC and dT thiophosphoramidites were synthesized as previous described (Yang, X-B., Fennewald, S., Luxon, B. A., Aronson, J., Herzog, N. and Gorenstein, D. G., “Aptamers containing thymidine 3′-O-phosphorodithioates: Synthesis and binding to Nuclear Factor-κB, J. Bioorganic and Medicinal Chemistry, 9, 3357-3362 (1999) and refs therein). The ODNs and DNs used in the study were synthesized on a 1-μmol scale on an Expedite 8909 System (Applied Biosystems) DNA Synthesizer.

Combinatorial selection of thioaptamers specific to TGF-β. Initial studies were focused on selection of thioaptamers specific to binding to TGF-β1. TGF-β1 is a 25 kDa homodimeric protein composed of two 12.5 kDa subunits joined by disulfide bonds (Roberts and Spom, 1993). TGF-β1 is secreted by immune cells and is instrumental in the initiation and resolution of inflammatory and immune events (Grotendorst, et al., 1989; Kehrl, et al., 1986). Blood monocytes respond to TGF-β1 by increased expression of inflammatory mediators such as IL-1, IL-6, and TNF-α (McCartney and Wahl, 1994). TGF-β1 acts as a chemotactic cytokine, attracting immune cells to the site of inflammation (Adams, et al., 1991). TGF-β1 overexpression has been demonstrated in prostate cancer (Truong, 1993) and in colon cancer (Zanetti, et al., 2003; Nagayama, et al., 2002).

Combinatorial selection of the thioaptamers disclosed was employed to isolate single-stranded DNA thioaptamers that specifically target the TGF-β1 protein. TGF-β1 protein is a homodimer, which imposes a structural requirement on candidate inhibitors; homodimeric forms of inhibitors are more effective in inhibiting the function of homodimeric proteins. Combinatorial selection of aptamers is advantageous in this respect, in that the selection isolates aptamers and thioaptamers that satisfy the structural requirement, unlike other methods of inhibitor selection such as the screening of small molecule libraries. This feature of the thioaptamer selection process will be discussed hereinbelow.

The initial thioaptamer selection process of the present invention was designed to modify the backbone of single-stranded DNA aptamers, with phosphoromonothioate substitutions 5′ of both A and C nucleotides. Thioation provides enhanced nuclease resistance as well as increased affinity and specificity relative to unmodified phosphate aptamers. Sequence data on the clones isolated during the selection, the predicted secondary structure of the clones, preliminary binding data and predicted dimeric models of the clones based on this data are described below.

The enabling technology used in the selection process, combinatorial selection and isolation of phosphorothioate DNA aptamers (thio-PCR, isolation of single strand DNA), analysis of DNA aptamer sequences and of target proteins, and evaluation of binding affinities of the selected DNA aptamers, has been covered in a series of patent applications and an issued patent of the primary inventor (U.S. Pat. No. 6,423,493; U.S. patent application Ser. No. 10/214,417; U.S. patent application Ser. No. 10/272,509; U.S. patent application Ser. No. 60/472,890), relevant portions incorporated herein by reference.

In one example, the present inventors have developed a thioaptamer targeting TGF-1, an important chemokine involved in the inflammatory response (Wahl, 1989, McCartney-Francis and Wahl, 1994). The TGFβ1 signaling pathway begins with secretion of TGF-β1 protein into the extracellular matrix along with auxiliary proteins, followed by TGF-β1 binding to its cell surface receptors. The cytoplasmic domain of one of the TGF-beta 1 receptors (TβRI) phosphorylates the transcription factors Smad2 and Smad3. The phosphorylated transcription factors are translocated into the nucleus by forming a heterodimer with Smad4, and there regulate target gene transcription (Shi, 2003).

As TGF-β1 is a homodimeric protein, its inhibitors may be dimeric forms in order to satisfy the symmetry requirement in binding. In order to find a dimeric inhibitor, one strategy is to first select a monomeric inhibitor and link two such monomers with a tether. Such a strategy may require that several tethers and variable tether lengths be tested in order to locate each unit of the inhibitor at its binding site in each subunit of the TGF-β1 protein. Flexibility of the tether must also be considered in developing an effective inhibitor of TGF-β1. If the tether is too flexible, binding of the dimeric inhibitor may be accompanied by a huge loss of entropy, reducing binding affinity. If the tether is too rigid, there may be no entropy loss upon binding, but tether length may have to be varied to locate the functional units of the inhibitor at the binding sites of the protein dimer. The combinatorial selection of aptamers described herein helps solve the symmetry problem as a consequence of its unique mechanism for selecting nucleic acid inhibitors (aptamers) from a randomized pool. As described hereinbelow, combinatorial aptamer and/or thioaptamer selection is expected to isolate aptamers that not only have TGF-β1 protein binding sites but also contain dimerization sites that can act as tethers. Consequently, the thioaptamers selected and isolated using combinatorial selection will be TGF-β1 protein inhibitors with high affinities.

Combinatorial selection of thioaptamers binding TGF-β1. A random, single-stranded combinatorial DNA library of normal phosporyl backbone oligonucleotides was synthesized by an automated DNA synthesizer that was programmed to include all four monomer bases of the oligonucleotide during the coupling of residues in a randomized segment. PCR primer segments at the 5′ and 3′ ends flanking the randomized region of the oligonucleotide of the synthesized library, were used in order to replicate and amplify the library with Taq DNA polymerase.

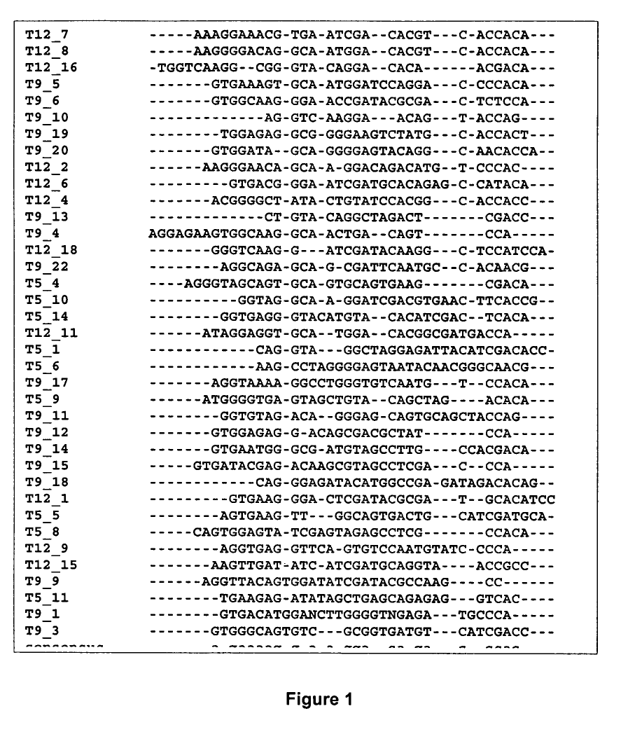

A 74-mer library was synthesized with a 30 base pair random central segment flanked by 23 and 21 base pair PCR primer regions. The single-stranded library was amplified using Taq polymerase and a mixture of dATP(αS), dCTP(αS), dGTP and dTTP. The PCR generated an oligonucleotide library with phosphorothioate backbone substituted at both dA and dC positions. Single-stranded DNA was isolated from the PCR products using streptavidin beads and the isolated single-stranded DNA was then incubated with TGF-β1 protein, and the protein-bound DNA was then isolated by filtration and that sequence was then subjected to the next round of selection and PCR amplification. This process was iterated repeatedly to isolate high affinity aptamer clones, as described in the thioaptamer clone listing and sequence alignment of FIG. 1. Following rounds 5, 9 and 12 of the iterative selection, the library was cloned and sequenced in order to monitor the selection. The sequences of the randomized regions of the selected clones were aligned using the Clustal W (1.8) algorithm, as shown in FIG. 1. Homology between sequences was observed using this alignment.

More particularly, a library was created as follows. A random single-stranded combinatorial DNA library of normal phosphoryl backbone oligonucleotides was synthesized by an automated DNA synthesizer (Midland Certified Reagents, Midland, Tex.) that was programmed to include all four monomer bases of the oligonucleotide during the coupling of residues in a randomized segment. In this example, PCR primer segments at the 5′ and 3′ ends are flanking the randomized region of the oligonucleotides of the synthesized library and thus the library can be replicated and amplified by Taq DNA polymerase (Amplitaq, Perkin-Elmer). A 74-mer library was used with a 30 base pair random central segment flanked by 23 and 21 base pair PCR primer regions: 5′-CAGTCCGGATGCTCTAGAGTGACN₃₀CGAATCTCGTGAAGCCGAGCG-3′ (SEQ ID NO.: 1). The diversity of the resulting library is theoretically be up to 43³⁰ (≅10¹⁸) different sequences. The single-stranded library was replicated using Klenow Fragment DNA polymerase and subsequently amplified using Taq polymerase. The oligonucleotide library with phosphorothioate backbone substituted at dA and dC positions was then synthesized by PCR amplification of the 74-mer template using commercially available Taq polymerase and a mixture of dATP (αS) (Amersham Biosciences), dTTP, dGTP and dCTP (αS) (Amersham Biosciences) as substrates. The PCR condition for amplification of the starting random library (5×10¹⁴ sequences) included: 200 μM each of dATP (αS), dTTP, dGTP, and dCTP (αS), 4 mM MgCl₂, 825 nM 74-mer random template, 50 units of Taq polymerase, and 2.4 μM each primer in a total volume of 1000 μL. PCR was performed to amplify the selected DNA with biotin-conjugated 5′ primer (biotin-biotin-biotin-5′-CAGTCCGGATGCTCTAGAGTGAC-3′ (SEQ ID NO.:2)) and 3′ primer (5′-CGCTCGGCTTCACGAGATTCG-3′ (SEQ ID NO.:3)) (Midland) under the following conditions: 94° C. for 5 min; 40 cycles at the 94° C. for 1 min, 65° C. for 2 min, and 72° C. for 3 min; the final extension was at 72° C. for 10 min. This polymerase acts stereospecifically to incorporate the Sp-diastereomers of dNTP (αS) and is believed to produce the Rp stereoisomer as is found for other polymerases (Eckstein, 1985).

Selection of Single-Stranded DNA Thioaptamers. A library of synthetic DNA oligonucleotides containing 30 random nucleotides flanked by invariant primer annealing sites was amplified by PCR using biotin-conjugated 5′ primer (biotin-biotin-biotin-5′-CAGTCCGGATGCTCTAGAGTGAC-3′ (SEQ ID NO.:2) and 3′ primer (5′-CGCTCGGCTTCACGAGATTCG-3′ (SEQ ID NO.:3)) (Midland Certified Reagents, Midland, Tex.). The 5′ primer had three biotin phosphoramidites covalently attached to its 5′ terminus. The 74-nucleotide double-stranded PCR product (˜1 nmol) was applied to 400 μL of a Magnetic Porous Glass (MPG) Streptavidin (CPG Inc.) (10 mg/ml, 4-6×10⁷ particles/ml) bead matrix suspended in Binding/Wash buffer (2.0 M NaCl, 1 mM EDTA, 10 mM Tris-HCl, pH 7.5). The mixture was rotated gently at room temperature for one hour. After equilibration of 1 hr at 20° C. to allow the biotinylated double-stranded DNA (dsDNA) to bind streptavidin beads, unbound dsDNA was removed with 900 μL of Binding/Wash buffer (2 times), and the matrix-bound dsDNA was denatured in 150 μL of Melting Solution (0.1 M NaOH) for 10 min at room temperature and washed one time with 150 μL of Melting Solution. As these conditions were not harsh enough to break the biotin-streptavidin interaction, this denaturation step released only nonbiotinylated single-stranded DNA strand from the bead complex (Bock, et al, 1992, and Schneider, et al, 1995). In order to remove NaOH in the sample and to collect the released nonbiotinylated single-stranded DNA, the supernatant of the sample was filtered with a Microcon YM-10 filter (Millipore) and washed with PBS buffer several times, yielding 0.1-0.3 nMol of single-stranded DNA (ssDNA).

The purity of the ssDNA library was confirmed by amplification of the correctly sized product with the reverse primer but not the forward primer after 5 cycles of PCR. Generally, 0.1˜0.3 nMol of the enriched single-stranded DNA was used for the following round of combinatorial selection. For the selection, this enriched single-stranded DNA was incubated with TGF-beta-1 protein (PeproTech, Rockhill, N.J.) in phosphate buffered saline (PBS) usually at room temperature for two hours and filtered through MF-Millipore nitrocellulose membrane filters (0.45 μm pore size, filter diameter 13 mm) presoaked previously in PBS. Under these conditions, the DNA/protein complexes were retained on the filter. The filter was then washed with 10 mL of PBS to remove the majority of the DNA, which only bound weakly to the protein. To elute the protein bound DNA the filter was incubated in elution solution containing 8 M urea at 65° C.˜75° C. for 10 min. In order to remove urea in the solution and to collect the protein-bound DNA, the supernatant of the sample was filtered with a Microcon YM-10 filter (Millipore) and washed with PBS buffer several times. The DNA retained on the filter was amplified by PCR to generate a DNA library for the next round selection. As the selection rounds proceeded, selection pressure was increased by reducing the protein concentration or increasing the salt concentration in the binding step (or both). DNA from the fifth, ninth, twelfth, and eighteenth rounds of selection, as well as the initial library, were cloned with the TOPO cloning kit (Invitrogen) and sequenced.

Analysis of Thioaptamer Sequences. The DNA sequences were obtained from various rounds of selection (5, 9, 12, and 18 round) and aligned using, e.g., the ClustalW algorithm (version 1.8), which is available from, e.g., a bioinformatics web site at Baylor College of Medicine (http://searchlauncher.bmc.tmc.edu). Secondary structure prediction of single-stranded DNA was conducted using, e.g., the mfold program (Zuker, et al., 2003), available at http://www.bioinfo.rpi.edu/applications/mfold/old/dna/form1.cgi.

Electrophoretic mobility shift assay (EMSA). The binding affinity of the TGF-beta-1 proteins to single-stranded DNA was analyzed using EMSA. The DNA sequence was 5′-CAGTCCGGATGCTCTAGAGTGAC-N₃₀-CGAATCTCGTGAAGCCGAGCG-3′ (SEQ ID NO.:1), which was biotin-labeled at the 3′ end using a Biotin 3′ End DNA Labeling Kit (Pierce) following the manufacture's protocol with few modifications. Before the labeling, ssDNA was boiled at 95° C. for 10 min to melt any secondary structures and quickly cooled by placing on ice. The labeling reaction was conducted at 37° C. for two hrs. After the biotin labeling, ssDNA was denatured and renatured by incubating it at 95° C. for 10 min and slowly cooling to room temperature with addition of MgCl₂ as final concentration to be 1 mM.

Next, 2 nM of biotinylated single-stranded DNA was incubated with variable amounts of TGF-beta-1 protein at room temperature for 30 min. Mixtures were loaded on native 10% polyacrylamide gels in 1× Tris Borate/EDTA electrophoresis buffer (TBE buffer)(Maniatis, et al., Molecular Cloning, CSH Press, NY (1989)), followed by electrophoresis at 8-10 V/Cm for 1 h. Nucleic acids in the gel were transferred to Biodyne nylon membranes (Pierce) by electroblotting at 100 V for 45 minutes using a Mini Trans-Blot Cell (Bio-Rad) and detected using LightShift Chemiluminescent EMSA kit (Pierce) following manufacture's protocol. A cooled CCD camera (Fluor Chem 8800 Imaging system) purchased from Alpha Innotech (San Leandro, Calif.) was used for image capture and measurement of IDV (Integrated Density Value) of chemiluminescent signals. Binding of the single-stranded DNA to TGF-beta-1 protein was assessed by measuring decrease of the chemiluminescence intensity values of free DNA as the protein was added to the reaction mixture.

FIG. 1 shows a Clustal W (1.8) alignment of sequences of clones isolated during the selection of thioaptamer binding TGF-β1 (through 12^{th} round of selection). The number preceding the underscore mark of the sequence identifier signifies the round of selection for each candidate thioaptamer and the number following the underscore mark signifies the clone number within that selection round. The blue and red characters within the oligonucleotide sequences signify moderate and highly conserved bases within the high affinity thioaptamers. For example, T12_{—}15 corresponds to the 15th thioaptamer clone selected on the 12^{th} round.

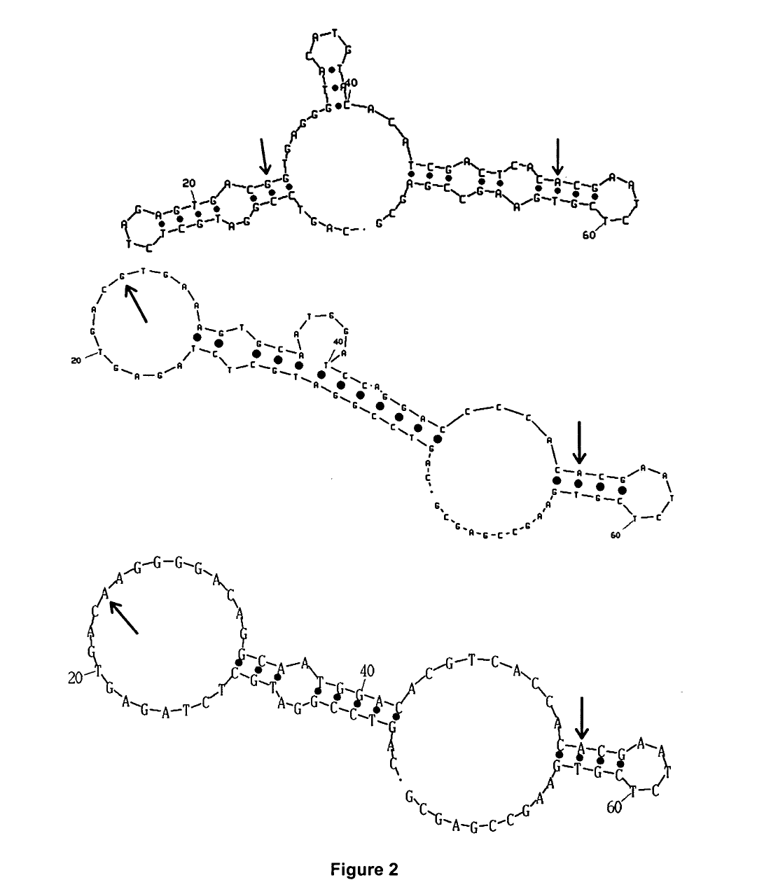

Secondary structures of the highest affinity sequence of the clones of each round (T5_{—}14, T9_{—}5, and T12_{—}8) were predicted using the mfold (3.1) algorithm. The predicted thioaptamer structures are shown in FIG. 2. Arrows indicate the first and last positions of nucleotides in the variable region. In all three thioaptamers, the sequences formed stable secondary structures with stem-loop motifs. Therefore, these secondary elements might be considered to be involved in binding and/or dimerization of the oligonucleotides. Another feature of the predicted secondary structures of the clones is the enrichment of phosphorothioates (A or C) between the two stems (e.g. between the second and third stems in the case of T5_{—}14). Given the fact that phosphorothioate modification improves binding affinity of a nucleic acid to proteins, this phosphorothioate-enriched region, observed in all three clones, can be hypothesized to be a major TGF-β1 binding site. Therefore, FIG. 2 shows the predicted secondary structures of highest affinity (to TGF-β1) thioapter clones of rounds 5, 9 and 12 (thioaptamers T5_{—}14, T9_{—}5, T12_{—}8). Next, an electromobility shift assay (EMSA) was run on the initial library and on the three high affinity thioaptamer clones selected from each of rounds 5 and 9.

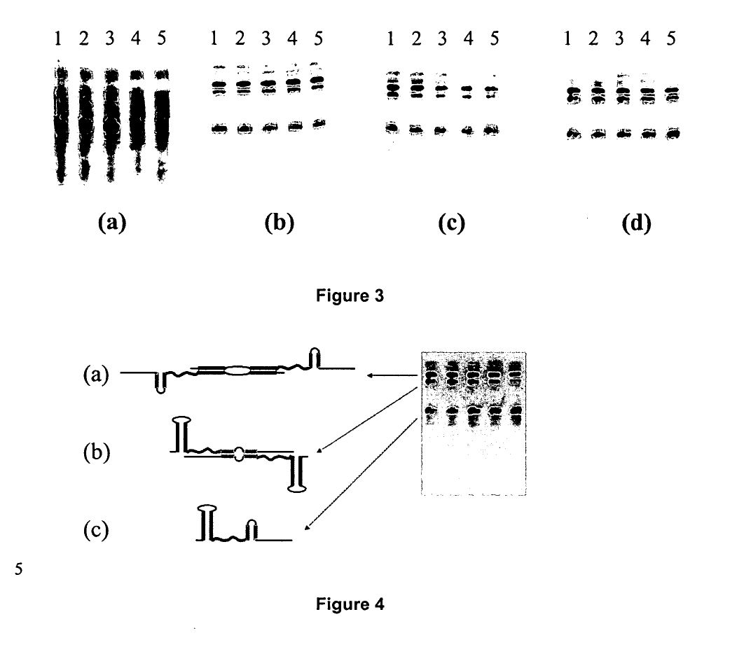

FIGS. 3a, 3b, 3c and 3d are gels of electromobility shift assays of the initial library (3a) and of thioaptamer candidates T5_{—}14, T9_{—}5 and T9_{—}22 (3b, 3c, 3d). EMSA was used to test the initial library and the three thioaptamer candidates for binding affinity to TGF-β1 protein. For the thioaptamer candidates, 2 nM biotinylated single-stranded DNA was incubated with 0 (lane 1), 9.8 nM (lane 2), 39.1 nM (lane 3), 156.3 nM (lane 4) and 625 nM of TGF-β1 protein for 30 minutes at room temperature. For the initial library, 2 nM biotinylated single-stranded DNA was incubated with 0 (lane 1), 500 nM (lane 2), 1000 nM (lane 3), 1500 nM (lane 4) and 2500 nM (Lane 5) of TGF-β1 protein for 30 minutes at room temperature.

Except for the initial library, each DNA thioaptamer candidate yielded three different bands in gel electrophoresis. These three bands were named top, middle and bottom bands, based on their electrophoretic mobility. Among these three bands, the bottom band corresponds to the main band of the initial library (as seen in FIG. 3), and it can thus be assigned to the monomeric form of the DNA (FIG. 2 structures). The molecular nature of the two other bands, top and middle, can be inferred based on the secondary structures predicted from their sequences. As shown in combination with FIG. 2, the predicted secondary structures contain at least two stem-loop regions. One of the stem-loops is predicted to be long and the other is predicted to be short. These stem-loop regions can provide the sites for the formation of homodimeric single-stranded DNA stabilized by intermolecular base pairings. Because there are two stem-loops, the DNA thioaptamer can form two different dimeric forms. The two upper bands (top and middle bands in FIG. 3)) showed a decrease in chemiluminescence intensity as TGF-β1 protein was added, indicating that those two forms of DNA bind to the protein. The bottom band showed a negligible decrease in chemiluminescence intensity at the protein concentrations used, indicating that it does not bind to the protein.

The molecular structures corresponding to the DNA bands observed in the EMSA gel assay were modeled based on the predicted secondary structures of the thioaptamer sequences, as shown in FIGS. 4a, 4b and 4c. DNA can exist as a monomeric form (FIG. 4c), or as two different dimeric forms (FIGS. 4a and 4b). In the monomeric form, there are two stem-loops, long and short ones (stems are presented as thick lines in FIG. 4a-4c). If the long stems undergo intermolecular base pairing so as to form a dimer, the dimer will assume an elongated form (as shown in FIG. 4a) which will have a low migration rate in the gel. If the short stem undergo intermolecular base pairing so as to form a dimer; the dimer will assume a more compact form with higher gel mobility than seen for the long stem-loop dimer (FIG. 4b). The squiggles in the DNA secondary structures of FIG. 4 represent phosphorothioate-rich regions located between the two stem-loops. The illustrative EMSA gel of FIG. 4 was that for the T9_{—}22 thioaptamer.

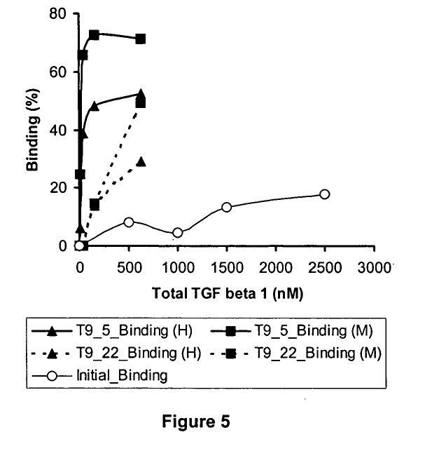

The decrease in chemiluminescence intensity of free DNA thioaptamer upon addition of TGF-β1 protein was used as a measure of the binding affinity of each thioaptamer to the target protein. The apparent binding constant, corresponding to a 50% decrease in the original chemiluminescence intensity of the band of the initial thioaptamer library (open circle in the plot of FIG. 5) was 11 uM. The apparent binding constant of clone T9_{—}5 thioaptamer is circa 150-480 nM for the top band (closed triangle and solid line) and ca. 50-200 nM for the middle band (closed square and solid line). The apparent binding constant of clone T9_{—}22 thioaptamer is circa 1.4 uM for the top band (closed triangle and dotted line) and 1.2 uM for the middle band (closed square and dotted line).

The monomeric forms of the thioaptamer candidates did not show any significant binding to target protein. The dimeric forms of T9_{—}5 and T9_{—}22 showed ca. 10-50 fold higher binding affinity to target protein relative to that of the initial library with the top bands having higher affinity than the middle band. It is therefore possible that dimeric forms of the thioaptamers are suitable candidates for target protein binding. This is consistent with the fact that the target protein itself, TGF-β1, is a homodimer and that in protein binding, satisfaction of the symmetry requirement should enhance the binding affinity of a nucleic acid.

TABLE 1
Apparent binding constants of thioaptamer candidates to TGF-β1

| | Apparent | | |
|---|---|---|---|
| | binding | | Improvement relative |
| Thioaptamer(s) | constant | | to initial library |
| Initial library | 11 | uM | — |
| T9_5 top band | 150-480 | nM | 23-73 fold |
| T9_5 middle band | 50-200 | nM | 55-200 fold |
| T9_22 top band | 1.4 | uM | 8-fold |
| T9_22 | 1.2 | uM | 9-fold |

FIG. 5 is a graph that shows binding of T9_{—}5, T9_{—}22 and initial library to TGF-β1 target protein. Based on the binding data depicted in FIG. 5, indicating that only dimeric forms of the thioaptamers bind to target protein, the predicted secondary structures of the selected thioaptamer candidates, and the modeling of candidate dimerization, the inventors have proposed a model for thioaptamer-target protein binding as shown in FIG. 6. It appears that two forms of the thioaptamer dimers, a compact form (left panel in FIG. 6) and an elongated form (right panel in FIG. 6) bind to TGF-β1 protein. The putative DNA-binding sites in the target protein are marked in dark color, however, the present invention is not limited in anyway by such markings. The phosphorothioate-enriched region of the thioaptamer is represented by a squiggle. The selection of single-stranded thioaptamer for TGF-β1 protein converged at round 18, as had been predicted using an algorithm developed by the present inventors. The convergence is shown in the Clustal W alignment listing of Table 2, below.

TABLE 2

Clustal W (1.8) alignment, penality = 20, of sequences of
clones isolated in 18th round selection of thioaptamers
binding TGF-β1:

| | | |
|---|---|---|
| T18_2_22 | 1 --TGTCGT--TGT--GTC--CTGTACCCG--CCTTGACCA- | (SED ID NO.: 4) |
| T18_1_3 | 1 --TGTCGT--TGT--GTC--CTGTACCCG--CCTTGACCA- | (SED ID NO.: 4) |
| T18_1_5 | 1 --TGTCGT--TGT--GTC--CTGTACCCG--CCTTGACCA- | (SED ID NO.: 4) |
| T18_1_6 | 1 --TGTCGT--TGT--GTC--CTGTACCCG--CCTTGACCA- | (SED ID NO.: 4) |
| T18_1_7 | 1 --TGTCGT--TGT--GTC--CTGTACCCG--CCTTGACCA- | (SED ID NO.: 4) |
| T18_1_12 | 1 --TGTCGT--TGT--GTC--CTGTACCCG--CCTTGACCA- | (SED ID NO.: 4) |
| T18_1_16 | 1 --TGTCGT--TGT--GTC--CTGTACCCG--CCTTGACCA- | (SED ID NO.: 4) |
| T18_1_20 | 1 --TGTCGT--TGT--GTC--CTGTACCCG--CCTTGACCA- | (SED ID NO.: 4) |
| T18_1_21 | 1 --TGTCGT--TGT--GTC--CTGTACCCG--CCTTGACCA- | (SED ID NO.: 4) |
| T18_2_17 | 1 --TGTCGT--TGT--GTC--CTGTACCCG--CCTTGACCA- | (SED ID NO.: 4) |
| T18_2_24 | 1 --TGTCGT--TGT--GTC--CTGTACCCG--CCTTGACCA- | (SED ID NO.: 4) |
| T18_1_2 | 1 TGTCTCGA--TGCTAGACT-CTATACCCG--CCAA------ | (SED ID NO.: 5) |
| T18_1_1 | 1 --TGTGGAC-TG---GTCT-ATCCATGCA--CCTGTACC-- | (SED ID NO.: 6) |
| T18_1_8 | 1 TGTGT-GTA-TG---GTCC-TTGCATCGATTCCCTG----- | (SED ID NO.: 7) |
| T18_2_13 | 1 --TGT-GTC-TA----TCG-CTGCACCGTGTCCAT-ACA-- | (SED ID NO.: 8) |
| T18_2_15 | 1 ---GT-GTG-CGTT-GTC---TGT--CCGTTTCCTGTCCAC | (SED ID NO.: 9) |
| T18_2_10 | 1 TGTGGCGT--TGAT-ATCGACTGT-------CCTTG-CCAC | (SED ID NO.: 10) |
| T18_2_1 | 1 TTGGTTGA--TGTGCATCG-CTGT-------TCCTGTCCA- | (SED ID NO.: 11) |
| T18_1_11 | 1 TTGGTTGA--TGTGCATCG-CTGT-------TCCTGTCCA- | (SED ID NO.: 11) |
| T18_1_4 | 1 TGGGTCGC--TG---ATC----GCATCGATACTCT--CCAC | (SED ID NO.: 12) |
| T18_1_24 | 1 ATCGTCGAC-TG----TC--CTGTCACTGT-CCAT--CCA- | (SED ID NO.: 13) |
| T18_1_9 | 1 --TGGAGG--TGCCTGGA---TATATC-GA--CTCGACCC- | (SED ID NO.: 14) |
| T18_1_14 | 1 --TGGAGG--TGCCTGGA---TATATC-GA--CTCGACCC- | (SED ID NO.: 14) |
| T18_1_22 | 1 --TGGAGG--TGCCTGGA---TATATC-GA--CTCGACCC- | (SED ID NO.: 14) |
| T18_2_2 | 1 --TGGAGG--TGCCTGGA---TATATC-GA--CTCGACCC- | (SED ID NO.: 14) |
| T18_2_3 | 1 --TGGAGG--TGCCTGGA---TATATC-GA--CTCGACCC- | (SED ID NO.: 14) |
| T18_2_4 | 1 --TGGAGG--TGCCTGGA---TATATC-GA--CTCGACCC- | (SED ID NO.: 14) |
| T18_2_5 | 1 --TGGAGG--TGCCTGGA---TATATC-GA--CTCGACCC- | (SED ID NO.: 14) |
| T18_2_6 | 1 --TGGAGG--TGCCTGGA---TATATC-GA--CTCGACCC- | (SED ID NO.: 14) |
| T18_2_7 | 1 --TGGAGG--TGCCTGGA---TATATC-GA--CTCGACCC- | (SED ID NO.: 14) |
| T18_2_9 | 1 --TGGAGG--TGCCTGGA---TATATC-GA--CTCGACCC- | (SED ID NO.: 14) |
| T18_2_12 | 1 --TGGAGG--TGCCTGGA---TATATC-GA--CTCGACCC- | (SED ID NO.: 14) |
| T18_2_14 | 1 --TGGAGG--TGCCTGGA---TATATC-GA--CTCGACCC- | (SED ID NO.: 14) |
| T18_2_18 | 1 --TGGAGG--TGCCTGGA---TATATC-GA--CTCGACCC- | (SED ID NO.: 14) |
| T18_2_19 | 1 --TGGAGG--TGCCTGGA---TATATC-GA--CTCGACCC- | (SED ID NO.: 14) |
| T18_2_23 | 1 --TGGAGG--TGCCTGGA---TATATC-GA--CTCGACCC- | (SED ID NO.: 14) |
| T18_1_10 | 1 -GTGTCCT--TGTCTAGCCTCGATA-------CACGACAC- | (SED ID NO.: 15) |
| T18_1_23 | 1 TGTGGCGT--TG----AC---TGTACACGTCGATACACC-- | (SED ID NO.: 16) |
| T18_1_19 | 1 --TGGCTGG-TCACTGTA--CTCT--CTGC-TCTCCAC--- | (SED ID NO.: 17) |
| T18_1_13 | 1 --TGTAGTG-TCC-TGGC---TATCCACGT--CTCCATT-- | (SED ID NO.: 18) |
| T18_1_18 | 1 ----TGGAT-CGCTTATCGCCTCGATC-----ATTGCCCA- | (SED ID NO.: 19) |
| T18_1_15 | 1 -TTGTTGTACTGGC-ATCGCCTCGACTCG---CTG------ | (SED ID NO.: 20) |
| Consensus | 1 tgt ga tg gtc ctgta c g ct gacc | (SED ID NO.: 21) |

In vivo testing of TGF-β thioaptamers. Cancer model—A thymic nude mouse will have tumor cells that overexpress TGF-β (human colon, breast, prostate, kidney cancer cells). Tumor cells will be implanted either orthotopically or ectopically and allowed to grow to 5×5×5 mm size. Animals will be then treated with TGF-β1 thioaptamer. Treatment may be done i.p., i.v., orally, s.c. or as an aerosol. The thioaptamer may also be packaged in different delivery systems such as liposomes or nanoparticles. In the orthotopic model, the effect upon angiogenesis, tumor invasion and metastasis may be determined. TGF-β is a potent inducer of angiogenesis and stimulates cell motility. Treatment with the TGF-β thioaptamer is expected to inhibit tumor growth, inhibit angiogenesis, and cause regression.

DMBA-induced breast cancer rat model will be used to examine the effect of TGF-β1 thioaptamer upon tumor progression as well as the immune system. TGF-β is a potent immune suppressor. The effects on tumor growth, metastasis, and immune functions (T and B cell function) will be examined in the presence and absence of TGF-β1 thioaptamer. In the cancer models, the TGF-β thioaptamer can be used in combination with other known therapeutic agents that enhance tumor regression or block tumor progression.

Fibrosis model—Transgenic mice that overexpress TGF-β in the liver develop not only liver but renal fibrosis (Mozes, et al., 1999; Kopp, et al., 1996, Terrel, et al., 1993). Mice may be treated with TGF-β1 thioaptamer at: 1 week, 3 weeks or 5 weeks of age. Mice are examined for the ability of TGF-β1 thioaptamer to block TGF-β stimulated liver and renal fibrosis. Alternatively, the effect of the TGF-β1 thioaptamer can be studied in a rat model in which TGF-β is administered for 14 days. These rats develop glomerulosclerosis (Terrell, et al., 1993). Wound Healing—Apply a skin wound to a mouse. This will be treated with or without a TGF-β1 thioaptamer. As a control, TGF-β will be applied topically in presence or absence of the thioaptamer.

### REFERENCES

D. H. Adams, et al., J Immunol, 147(2), 609-612 (1991)—Transforming growth factor-beta induces human T lymphocyte migration in vitro.
Y. Akiyama, et al., Gastroent, 112, 33-39 (1997)—Transforming growth factor-beta type II receptor gene mutations in adenomas for hereditary nonpolyposis colon cancer.
M. A. Anzano, et al., Cancer Res, 49, 2898-2904 (1989)—Growth factor production by human colon carcinoma cell lines.
L. C. Bock, et al., (1992) Selection of single-stranded DNA molecules that bind and inhibit human thrombin. Nature, 355, 564-566.
E. P. Bottinger, et al., PNAS, 93(12), 5877-5882 (1996)—The recombinant proregion of transforming growth factor beta (latency-associated peptide) inhibits active transforming growth factor beta 1 in transgenic mice.
F. T. Boyd and J. Massague, J Biol Chem, 264, 2272-2278 (1989)—Transforming growth factor-beta inhibition of epithelial cell proliferation linked to expression of 53 kDa membrane receptor.
T. Brabletz, et al., Mol Cell Biol, 13, 1155-1162 (1993)—Transforming growth factor β and cyclosporin A inhibit the inducible activity of the interleukin-2 gene in T cells through a noncanonical octamer-binding site.
J. K. Burmester, et al., PNAS, 90(18), 8628-8632 (1993)—Characterization of distinct functional domains of transforming growth factor beta.
T. Chen, et al., Cancer Res, 58, 48054810 (1998)—Transforming growth factor-β type I receptor kinase mutant associated with metastatic breast cancer.
F. Cognasse, et al., Clin Exp Immunol, 132(2), 304-308 (2003)—HIV-gp160 modulates differentially the production in vitro of IgG, IgA and cytokines by blood and tonsil B lymphocytes from HIV-negative individuals.
Eckstein, F., Annu. Rev. Biochem. 54, 367402 (1985). Nucleoside phosphorothioates.
A. D. Ellington and J. W. Szostak, (1990) In vitro selection of RNA molecules that bind specific ligands. Nature, 346, 818-822.
H. Fakhrai, et al., PNAS, 93(7), 2909-2914 (1996)—Eradication of established intracranial rat gliomas by transforming growth factor beta antisense gene therapy.
M. L. Garda, et al., J Immunol, 168(5), 2247-22564 (2002)—HIV antigens can induce TGF-beta(1)-producing immunoregulatory CD8+ T cells.
A. G. Geiser, et al., Gene, 129(2), 223-228 (1993)—Regulation of the transforming growth factor-beta 1 and -beta 3 promoters by transcription factor Sp1.
J. George, et al., PNAS, 96(22), 12719-12724 (1999)—In vivo inhibition of rat stellate activation by soluble transforming growth factor beta type II receptor: a potential new therapy for hepatic fibrosis.
M. Goggins, et al., Cancer Res, 58, 5329-5332 (1998)—Genetic alterations of the transforming growth factor-beta receptor genes in pancreatic and biliary adenocarcinomas.
L. Gold, et al. (1997) SELEX and the evolution of genomes, Curr. Opin. Genetic. Dev., 7, 848-851.
L. Gorelik and R. A. Flavell, Nat Rev Immunol, 2(1), 46-53 (2002)—Transforming growth factor-beta in T-cell biology.
E. A. Grimm, et al., Cancer Immunol Immunother, 27(1), 53-58 (1988)—TGF-beta inhibits in vitro induction of lymphokine-activated killing activity.
G. R. Grotendorst, et al., J Cell Physiol, 140(2), 396402 (1989)—Production of tranforming growth factor beta by human peripheral blood monocytes and neutrophils.
P. J. Hart, et al., Nat Struct Biol, 9, 203-208 (2002)—Crystal structure of the human ThetaR2 ectodomain-TGF-beta3 complex.
Y. Hasegawa, et al., Cancer, 91(5), 964-971 (2001)—Transforming growth factor-beta 1 level correlates with angiogenesis, tumor progression, and prognosis in patients with nonsmall cell lung carcinoma.
P. Hau, et al., American Society of Clinical Oncology, 2002 meeting, abstract 109—TGF-beta 2 antisense oligonucleotide AP 12009 administered intratumorally to patients with malignant glioma in a clinical phase I/II dsose escalation study: safety and preliminary efficacy data.
A. P. Hinck, et al., Biochemistry, 35(26), 8517-8534 (1996)—Transforming growth factor beta 1: three-dimensional structure in solution and comparison with the X-ray structure of transforming growth factor beta 2.
M. Huse, et al., Cell, 96, 425436 (1999)—Crystal structure of the cytoplasmic domain of the type 1 TGF-beta receptor in complex with FKBP 12.
N. Ito, et al., Cancer Lett, 89(1), 4548 (1995)—Positive correlation of plasma transforming growth factor-beta 1 with tumor vascularity in hepatocellular carcinoma.
P. Jachimczak, et al., Proceedings of the 82 nd annual meeting of the Am Assoc Cancer Res, Houston, Tex., May 15-18, 1991, 32 (O), 427, 1991.
P. Jachimczak, et al., J Neurosurg, 78(6), 944-951 (1993)—The effect of transforming growth factor-beta-2-specific phosphorothioate-anti-sense oligodeoxynucleotides in reversing cellular immunosuppression in malignant glioma.
P. Jachimczak, et al., Cellular Immunol, 168, 125-133 (1995)—Tranforming growth factor-β-mediated regulation of human peripheral blood mononuclear cell proliferation as detected with phosphorothioate antisense oligodeoxynucleotides.
P. Jachimczak, et al., Int J Cancer, 65(3), 332-337 (1996)—Transforming growth factor-beta mediated autocrine growth regulation of gliomas as detected with phosphorothioate antisense oligonucleotides.
J. Y. Kao, et al., J Immunol, 170(7), 3806-3811 (2003)—Tumor-derived TGF-beta reduces the efficacy of dendritic cell/tumor fusion vaccines.
J. H. Kehrl, et al., J Exp Med, 163(5), 1037-1050 (1986)—Production of transforming growth factor beta by human T-lymphocytes and its potential role in the regulation of T-cell growth.
M. Koizumi and R. R. Breaker, (2000) Molecular recognition of cAMP by an RNA aptamer, Biochemistry, 39, 8983-8992.
Kong, Ann Surg, 222(2), 155-162 (1995)—Elevated plasma TGF-β1 levels in breast cancer patient decrease after surgical removal of tumor.
J. B. Kopp, et al., Lab Invest, 74, 991-1003 (1996)—Transgenic mice with increased plasma levels of TGF-β1 develop progressive renal disease.
T. Kossman, et al., J Infect Dis, 187(4), 534-541 (2003)—Cytomegalovirus production by infected astrocytes correlates with transforming growth factor-beta release.
M. C. Kuppner, et al., J Neurosurg, 71(2), 211-217 (1989)—Inhibition of lymphocyte function by glioblastoma-derived transforming growth factor beta 2.
R. G. Lima, et al., J Infect Dis, 185(11), 1561-1566 (2002)—The replication of human immunodeficiency virus type 1 in macrophages is enhanced after phagocytosis of apoptotic cells.
J. MacCallum, et al., Br J Cancer, 69, 1006-1009 (1994)—Expression of transforming growth factor beta mRNA isoforms in human breast cancer.
G. Manning, et al., Science, 298, 1912-1934 (2002)—The protein kinase complement of the human genome.
S. Markowitz, et al., Science, 268, 1336-1338 (1995)—Interaction of the type II TGF-β receptor in colon cancer cells with microsatellite instability.
J. Massague, Ann Rev Biochem, 67, 753-791 (1998)—TGF-beta signal transduction.
J. Massague, et al., Cell, 103, 295-309 (2000)—TGF-beta signaling in growth control, cancer, and heritable disorders.
N. L. McCartney-Francis and S. M. Wahl, J Leukocyte Biol, 55(3), 401-409 (1994)—Transforming growth factor beta: a matter of life and death (review).
Mozes M. M. et al. J Am Soc Nephrol 10, 271-280 (1999)—Renal expression of fibrotic matrix proteins and of transforming growth factor-β (TGF-β) isoforms in TGF-β transgenic mice.
S. Nagayama, et al., Anticancer Res, 22(6B), 3545-3554 (2002)—Altered expression of the receptor and ligand in the TGF beta signaling pathway in differential infiltrating colon carcinoma.
S. W. Qian, et al., J Biol Chem, 271(48), 30656-62 (1996)—Binding affinity of transforming growth factor-β for its type II receptor is determined byu the C-terminal region of the molecule.
S. W. Qian, et al., Growth Factors, 17(1), 63-73 (1999)—Distinct functional domains of TGF-β bind receptors on endothelial cells.
A. B. Roberts and M. B. Sporn, Growth Factors, 8, 177, (1993)—Physiological activities and clinical applications of TGF-β.
H. Robson, et al., Brit J Cancer, 74(5), 753-758 (1996)—TGF-β1 expression in human colorectal tumors: an independent prognostic marker in a subgroup of poor prognosis patients.
S. A. Salzman, et al., Cytokines Mol Ther, 7(1), 31-36 (2002)—Regulation of colony-stimulating factor-induced human myelopoiesis by TGF-β isoforms.
Roswell Park Cancer Institute website, Oct. 13, 2003—Program: Structure/activity studies of TGF-beta.
Y. Sawamura and N. de Tribolet, Adv Tech Stand Neurosurg, 17, 3-64 (1990)—Immunobiology of brain tumors.
W. P. Schiemann, et al., Blood, 94, 2854-2861 (1999)—A deletion in the gene for transforming growth factor-beta type I receptor abolishes growth regulation by TGF-beta in a cutaneous T-cell lymphoma.
H. Schluessner, et al., J Neuroimmunol, 28, 271-276 (1990)—Susceptibility and resistance of human automimmune T cell activation to the immunoregulatory effects of transforming growth factor β1, β2 and β1.2.
Schneider, et al., Biochemistry. 34, 9599-9610 (1995). High-affinity ssDNA inhibitors of the reverse transcriptase of type 1 human immunodeficiency virus.
D. Schuppan, et al., Cell Death Differ, 10 Suppl 1:S59-67(2003)—Hepatitis C and liver fibrosis.
Scios website, TGF-β Inhibitor Program, Oct. 13, 2003.
M. Shah, et al., The Lancet, 339, 213-214 (1992)—Control of scarring in adult wounds by neutralizing antibody to transforming growth factor β.
Y. Shi, et al., Cell, 113, 685-700 (2003)—Mechanisms of TGF-β signaling from cell membrane to nucleus.
M. B. Sporn, et al., Science, 219, 1329-1331 (1983)—Polypeptide transforming growth factor isolated from bovine sources and used for wound healing in vivo.
M. Suh, et al., Cancer Res, 63(6), 1371-1376 (2003)—Synthetic triterpenoids enhance transforming growth factor beta/Smad signaling.
P. D. Sun and D. R. Davies, Annu Rev Biophys Biomol Struct, 24, 269-291 (1995)—The cysteine-knot growth factor superfamily.
S. L. Swain, et al., J Immunol, 147, 2991-3000 (1991)—Transforming growth factor β and IL4 cause helper T precursors to develop into distinct effector helper cells that differ in lymphokine secretion pattern and cell surface phenotype.
T. G. Terrell, et al., Int Rev Exp Pathol, 34B, 43-67 (1993)—Pathology of recombinant human transforming growth factor-β1 in rats and rabbits.
L. D. Truong, et al., Hum Pathol, 24, 4-9 (1993)—Association of transforming growth factor beta 1 with prostate cancer: an immunohistochemical study.
T. Tsukazaki, et al., Cell, 95, 779-791 (1998)—SARA, a FYVE domain protein that recruites Smad2 to the TGF-beta receptor.
H. Tsushima, et al., Gastroenterol, 110(2), 375-382 (1996)—High levels of TGF-β1 in patients with colorectal cancer: association with disease progression.
Vanderbilt University website, 10-13-03, Modulation of TGF-beta signaling vy STRAP.
S. M. Wahl, et al., Immunol Today, 10(8), 258-261 (1989)—Inflammatory and immunomodulatory roles of TGF-beta.
J. A. Vrana, et al., Cancer Res, 56(21), 5063-5070 (1996)—Expression of tissue factor in tumor stroma correlates with progression to invasive human breast cancer: paracrine regulation by carcinoma cell-derived members of the TGF-β family.
D. Wang, et al., Cancer Res, 60, 4507-4512 (2000)—Analysis of specific gene mutations in the transforming growth factor-β signal transduction pathway in human ovarian cancer.
Q. Wang, et al., Thorax, 54, 805-812 (1999)—Reduction of bleomycin-induced lung fibrosis by transforming growth factor beta soluble receptor in hamsters.
J. Wang, et al., J Immunol, 167(6), 3360-3366 (2001)—Synergistic induction of apoptosis in primatry CD4(+) T cells by macrophage-tropic HIV-1 and TGF-beta 1.
G. Wu, et al., Science, 287, 92-97 2000)—Structural basis of Smad2 recognition by the Smad anchor for receptor activation.
Y. Yamaguchi, et al., Nature, 346, 281-284 (1990)—Negative regulation of tranforming growth factor-β, by the proteoglycan decorin.
X. Ye, et al. (1996) Deep penetration of an α-helix into the widened RNA major groove in the HIV-1 Rev peptide-RNA aptamer complex, Nat. Struct. Biol., 3, 1026-1033.
D. Zanetti, et al., Mol Aspects Med, 24(4-5), 273-280 (2003)-4-hydroxynonenal and transforming growth factor beta 1 expression in colon cancer.
Zuker, M., Nucleic Acid Research, 31(13): 3406-3415 (2003), Mfold web server for nucleic acid folding and hybridization prediction.
   Patents
U.S. Pat. No. 6,509,318, TGF-β Inhibitors and methods, R. S. Bhatnagar, et al., issued Jan. 21, 2003.
U.S. Pat. No. 6,455,689 B1, Antisense oligonucleotides for transforming growth factor-β, G-F. Schlingensiepen, et al., issued Sep. 24, 2002.
U.S. Pat. No. 6,447,769, Compositions and methods for enhanced tumor cell immunity in vivo, H. Fakhrai, et al., issued Sep. 10, 2002.
U.S. Pat. No. 6,423,493, Combinatorial selection of oligonucleoside aptamers, D. Gorenstein, et al., issued Jul. 23, 2002.
U.S. Pat. No. 6,201,108, TGF-β type receptor cDNAs, encoded products and uses thereof, H. Y. Lin et al, issued Mar. 13, 2001.
U.S. Pat. No. 6,120,763, Compositions and methods for enhanced tumor cell immunity in vivo, H. Fakhrai, et al., issued Sep. 19, 2000.
U.S. Pat. No. 6,086,867, Modulation of TGF-β by TGF-β type III receptor polypeptides, H. Y. Lin et al, issued Jul. 11, 2000.
U.S. Pat. No. 5,772,995, Compositions and methods for enhanced tumor cell immunity in vivo, H. Fakhrai, et al., issued Jun. 30, 1998.
U.S. Pat. No. 5,683,988, Anti-sense oligodeoxynucleotide to fibrotic cytokine TGF-β and uses thereof, H. T. Chung, issued 114-97.
U.S. Pat. No. 5,662,904, Anti-scarring compositions comprising growth factor neutralizing antibodies, M. W. J. Ferguson, et al., issued Sep. 2, 1997.
   Patent Applications

U.S. Patent Application 20030040499, Antisense-oligonucleotides for the treatment of immuno-suppressive effects of transforming growth factor-beta (TGF-beta), G-F. Sclingensiepen, et al., published Feb. 27, 2003.

1. A partially thio-modified aptamer that binds to a TGF-beta protein.

2. The aptamer of claim 1, wherein the TGF-beta protein comprises a human TGF-beta.

3. The aptamer of claim 1, wherein the TGF-beta protein comprises a TGF-beta dimer.

4. The aptamer of claim 3, wherein the TGF-beta dimer is a homodimer.

5. The aptamer of claim 4, wherein the TGF-beta homodimer is a TGF-beta 1, 2 or 3 homodimer.

6. The aptamer of claim 3, wherein the TGF-beta dimer is a TGFbeta 1, 2 or 3 heterodimer.

7. The aptamer of claim 1, wherein the aptamer comprises one or more thio-modifications as set forth in SEQ ID NOS: 4-22.

8. The aptamer of claim 1, wherein the aptamer is achiral.

9. The aptamer of claim 1, wherein the aptamer further comprises a detectable label.

10. The aptamer of claim 1, further comprising one or more pharmaceutically acceptable salts.

11. The aptamer of claim 1, further comprising a diluent.

12. A partially thio-modified aptamer that binds to a TGF-beta receptor.

13. The aptamer of claim 12, wherein the TGF-beta receptor is a signaling receptor.

14. The aptamer of claim 12, wherein the TGF-beta receptor is a co-receptor.

15. The aptamer of claim 13, wherein the TGF-beta signaling receptor comprises a human TGF-beta signaling receptor.

16. The aptamer of claim 13 wherein the TGF-beta signaling receptor comprises a ThetaRI or a TbetaRII receptor.

17. The aptamer of claim 13, wherein the target of the aptamer is the GS domain of a ThetaRI receptor.

18. The aptamer of claim 14, where the co-receptor is TGF-beta 3.

19. The aptamer of claim 12, wherein the aptamer is achiral.

20. A partially thio-modified aptamer that binds to a ligand-receptor complex comprising a TGF-beta ligand and a receptor complex comprising a ThetaRI and a ThetaRII receptors.

21. The aptamer of claim 20, wherein the target of the aptamer is the GS domain of a ThetaRI receptor.

22. The aptamer of claim 20, wherein the aptamer is achiral.

23. A partially thio-modified aptamer that binds to a ligand binding trap capable of trapping TGF-beta ligands.

24. The aptamer of claim 23, wherein the ligand binding trap comprises decorin, latency-associated protein (LAP) or alpha-macroglobulin.

25. The aptamer of claim 23, wherein the aptamer is achiral.

26. A partially thio-modified aptamer that binds to an auxiliary protein that promotes binding of TGF-beta ligand to Theta signaling receptors.

27. The aptamer of claim 26, wherein the auxiliary protein is a SARA protein.

28. The aptamer of claim 26, wherein the aptamer is achiral.

29. A partially thio-modified aptamer that binds to a Smad protein.

30. The aptamer of claim 29, wherein the Smad protein is an R-Smad, a Co-Smad, an I-Smad or a combination thereof.

31. The aptamer of claim 29, wherein the aptamer is achiral.

32. A partially thio-modified aptamer that binds to a TGF-beta protein complex and enhances TGF-beta activity.

33. The aptamer of claim 32, wherein the binding site of the aptamer on the TGF-beta protein complex comprises a region of a ligand binding trap protein.

34. The aptamer of claim 32, wherein the binding site of the aptamer on the TGF-beta protein complex comprises a region of an inhibitory I-Smad.

35. The aptamer of claim 32, wherein the aptamer is achiral.

36. A partially thio-modified aptamer that binds to a TGF-beta protein complex and inhibits TGF-beta activity.

37. The aptamer of claim 36, wherein the binding site of the aptamer on the TGF-beta protein complex comprises a region of an R-Smad or a Co-Smad.

38. The aptamer of claim 36, wherein the aptamer is achiral.

39. A partially modified thioaptamer that inhibits TGF-beta activity by binding to a TGF-beta ligand, a TGF-beta ligand-Theta receptor complex, a TGF-beta signaling receptor and co-receptor, to an R-Smad or a Co-Smad.

40. The aptamer of claim 39, wherein the aptamer is achiral.

41. A partially modified thioaptamer that modifies TGF-beta activity by binding to a TGF-beta ligand, a TGF-beta ligand-Theta receptor complex, a TGF-beta signaling receptor and co-receptor, to an R-Smad or a Co-Smad.

42. A method of inhibiting TGF-β activity comprising the steps of:
providing to a host in need of therapy a pharmaceutically effective amount of a thioaptamer that specifically binds to and inhibits TGF-β activity.

43. The method of claim 42, wherein the thioaptamer is provided to the host to ameliorate the effects of: fibrosis, scarring and adhesion during wound healing; fibrotic diseases of the lung, liver and kidney; atherosclerosis, arteriosclerosis; cancers including gliomas, colon cancer, prostate cancer, breast cancer, neurofibromas, lung cancer; angiopathy, vasculopathy, nephropathy; systemic sclerosis; viral infections accompanied by immune suppression (HIV, HCV); and immunological disorders and deficiencies (auto-immune diseases).

44. A method of quantitating TGF-β levels in a sample comprising the step of contacting a sample with a TGF-β-specific thioaptamer.

45. The method of claim 44, wherein the samples comprises a physiological sample.

46. The method of claim 44, wherein the sample comprise a blood, tissue, cells, supernatant, media.

47. The method of claim 44, wherein the TGF-β protein comprises a human TGF-β.

48. The method of claim 44, wherein the TGF-β protein comprises a TGF-β homodimer.

49. The method of claim 44, wherein the TGF-β protein comprises a TGF-β1, 2 or 3 heterodimer.

50. The method of claim 44, wherein the thioaptamer comprises one or more thio-modifications as set forth in SEQ ID NOS.: 4-22.

51. The method of claim 44, wherein the thioaptamer further comprises a detectable label.

52. The method of claim 44, wherein the thioaptamer further comprises a detectable detectable selected from the group consisting of a calorimetric, a fluorescent, a radioactive and an enzymatic agent.

53. A method of modulating TGF-β signaling comprising the steps of:
administering to a host a TGF-β specific thioaptamer that modulates the activity through the TGF-β receptor in a dosage effective to reduce activity of the TGF-β.

54. The method of claim 53, wherein the thioaptamer modulates the activity through the TGF-β receptor by increasing activity.

55. The method of claim 53, wherein the thioaptamer modulates the activity through the TGF-β receptor by decreasing activity.

56. The method of claim 53, wherein the thioaptamer is selected from the group consisting of SEQ ID NOS.:4-22.

57. A method of treating a pathological condition due to increased TGF-β activity comprising the steps of:
administering to a host an effective dosage of a thioaptamer that modulates TGF-β.

58. The method of claim 57, wherein the thioaptamer binds to TGF-β, the TGF-β receptor, a TGF-β auxiliary protein, a TGF-β, ligand binding trap protein or a TGF-β Smad protein.

59. The method of claim 57, wherein the thioaptamer modulates the activity through the TGF-β receptor by increasing activity.

60. The method of claim 57, wherein the thioaptamer modulates the activity through the TGF-β receptor by decreasing activity.

61. The method of claim 57, wherein the thioaptamer is selected from the group consisting of SEQ ID NOS.: 4-22.

62. The method of claim 57, wherein the pathological condition comprises:
fibrosis, scarring and adhesion during wound healing; fibrotic diseases of the lung, liver and kidney; atherosclerosis and arteriosclerosis; cancers such as gliomas, colon cancer, prostate cancer, breast cancer, neurofibromas, lung cancer; angiopathy, vasculopathy, nephropathy;
systemic sclerosis; viral infections accompanied by immune suppression (HIV, HCV); and
immunological disorders and deficiencies (auto-immune diseases).

63. The method of claim 57, wherein the TGF-β specific thioaptamer is encapsulated.

64. The method of claim 57, wherein the capsule is degradable by an external stimulus to release the TGF-β specific thioaptamer.

65. The method of claim 57, wherein the external stimulus is selected from the group consisting of UV light, acid, water, in vivo enzymes, ultrasound and heat.

66. The method of claim 57, wherein the TGF-βspecific thioaptamer is bound to a binding molecule.

67. The method of claim 57, wherein the TGF-β specific thioaptamer is bound to a binding molecule and further comprising the step of detaching the binding molecule from the TGF-β specific thioaptamer.

68. A method of treating a pathological condition in which increased TGF-β activity has been implicated comprising the steps of:
administering to a host a TGF-β specific thioaptamer in a pharmaceutically acceptable carrier at a dosage effective to reduce TGF-β activity.

69. The method of claim 68, wherein the pharmaceutically acceptable carrier is selected from the group consisting of a cream, gel, aerosol and powder for topical application.

70. The method of claim 68, wherein the pharmaceutically acceptable carrier is selected from the group consisting of a sterile solution for injection, irrigation and inhalation.

71. The method of claim 68, wherein the pharmaceutically acceptable carrier comprises a sterile dressing for topically covering a wound.

72. The method of claim 68, wherein the pharmaceutically acceptable carrier is selected from the group consisting of a biopolymer and a polymer for implanting within a wound.

73. The method of claim 68, further comprising the step of administering a growth factor other than TGF-β.

74. The method of claim 68, wherein the TGF-β specific thioaptamer is encapsulated.

75. A method of modulating TGF-β signaling comprising the steps of:
administering to a host a TGF-β3 ligand binding trap specific thioaptamer that modulates the activity through the TGF-β receptor in a dosage effective to reduce activity of the TGF-β.

76. A method of modulating TGF-β signaling comprising the steps of:
administering to a host a TGF-β auxiliary protein specific thioaptamer that modulates the activity through the TGF-β receptor in a dosage effective to reduce activity of the TGF-β.

77. A method of modulating TGF-β signaling comprising the steps of: administering to a host a TGF-β Smad protein specific thioaptamer that modulates the activity through the TGF-β receptor in a dosage effective to reduce activity of the TGF-β.
